(19) United States
(12) Patent Application Publication
(10) Pub. No.: US 2022/0112208 A1
(43) Pub. Date: Apr. 14, 2022

(54) COMPOUNDS WITH ANTIINFLAMMATORY ACTIVITY AND METHODS OF USE THEREOF

(71) Applicants: King Abdullah University of Science and Technology, Thuwal (SA); National and Kapodistrian University of Athens, Athens (GR); University of Crete, Crete (GR)

(72) Inventors: Carlos M. Duarte, Thuwal (SA); Susana Agusti, Thuwal (SA); Mariusz Jaremko, Thuwal (SA); Lukasz Jaremko, Thuwal (SA); Vaileios Roussis, Athens (GR); Efstathia Ioannou, Athens (GR); Aikaterini Koutsaviti, Athens (GR); Christos Tsatsanis, Heraklion P.C. (GR); Sotirios Kampranis, Heraklion P.C. (GR); Maria Daskalaki, Heraklion P.C. (GR)

(21) Appl. No.: 17/497,649

(22) Filed: Oct. 8, 2021

Related U.S. Application Data
(60) Provisional application No. 63/089,059, filed on Oct. 8, 2020.

(51) Int. Cl. C07D 493/04; C07D 493/14

(52) U.S. Cl. CPC C07D 493/04; C07D 493/14

(57) ABSTRACT

Compounds with anti-inflammatory activity, methods of extracting and isolating the compounds from seaweed, and methods of using the compounds are disclosed herein. The compounds can be extracted and isolated from seaweed, such as Laurencia. Typically, the compound disclosed herein has anti-inflammatory activity with negligible toxicity, and thus can be used as anti-inflammatory agents.

This application claims the benefit of and priority to U.S. Provisional Application No. 63/089,059, filed Oct. 8, 2020, which is hereby incorporated herein by reference in its entirety.

### FIELD OF THE INVENTION

The invention is generally directed to compounds with anti-inflammatory activity.

### BACKGROUND OF THE INVENTION

Inflammatory problems underlay a broad range of health systems, from epidermal to internal, with many of the pharma products available being toxic, synthetic and carrying important side effects. The pharma, cosmetic, skin care and nutraceutical sectors are searching for natural compounds that can be produced naturally, rather than synthesized in the lab.

The genus Laurencia (Rhodomelaceae) is a cosmopolitan genus, comprising c. 180 accepted species [1]. Red algae of this genus occur mainly in temperate, subtropical and tropical coastal environments, littoral to sublittoral, throughout the world, down to 65 m depth [1]. The taxonomy of the genus has undergone important revisions and it still is a subject of debate, due to the diversity and/or the plasticity of the markers used for the distinction of taxa. Apart from the challenging taxonomy, the genus presents a wide chemical diversity and an unparalleled ability to produce a large variety of secondary metabolites, including C₁₅ acetogenins, sesquiterpenes, diterpenes and triterpenes, often with a high degree of halogenation, offering conferring to the organism effective chemical defense against herbivores [2,3].

There remains a need for compounds from the seaweed, such as Laurencia sp., with anti-inflammatory activities.

Therefore, it is the object of the present invention to provide compounds with anti-inflammatory activities.

It is another object of the present invention to provide methods of making the compounds with anti-inflammatory activities.

It is yet another object of the present invention to provide methods of using the compounds with anti-inflammatory activities.

### SUMMARY OF THE INVENTION

Compounds with anti-inflammatory activities, methods of making the compounds, and methods of using the compounds as anti-inflammatory agents are disclosed herein.

The compound can have a structure of Formula (I).

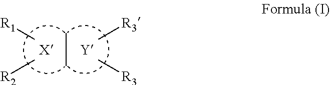

where X′ is a C₄ five-membered heterocyclic group or a C₅ six-membered heterocyclic group; Y′ is a C₄ five-membered heterocyclic group, a C₅ six-membered heterocyclic group, or a C₇ eight-membered heterocyclic group; R₁ and R₂ are independently absent, a halogen, or a substituted or unsubstituted alkyl group; R₃ and R₃′ are independently absent, a halogen, a hydroxyl group, a thiol group, an amino group,

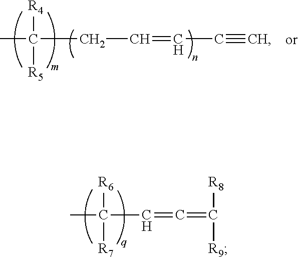

R₄-R₉ are independently a hydrogen, a halogen (e.g. a fluorine, a chlorine, a bromine, or an iodine, such as bromine or chlorine), an hydroxyl group, a thiol group, or an amino group; each of m, n, and q is an integer from 0 to 3; and when R₁ and/or R₂ are independently a substituted alkyl group, the substituent is a halogen (e.g. a fluorine, a chlorine, a bromine, or an iodine, such as bromine or chlorine), an hydroxyl group, a thiol group, or an amino group.

In some preferred embodiments, the compound has a structure of any one of compounds a1-a8 described below.

Typically, the compounds disclosed herein have anti-inflammatory activity with negligible toxicity, and can be used as anti-inflammatory agents in, for example, food products, cosmetics products, skin care products, nutraceuticals and pharmaceuticals for humans, as well as in veterinary products. For example, the compounds may be used in a method for preventing, treating, or ameliorating one or more symptoms associated with an inflammation in a subject are disclosed.

Generally, the method of using the disclosed compounds includes (i) administering to the subject an effective amount of the compound(s) to prevent, treat, or ameliorate one or more symptoms associated with inflammation in the subject. The subject can be a mammal. The compound(s) can be administered by a medical professional or the subject being treated (e.g. self-administration). In some embodiments, the compound(s) is formulated in a formulation or composition with a suitable excipient and is administered in the form of the formulation or composition in the subject.

The compounds can be extracted and isolated from seaweed, such as Laurencia sp. In some embodiments, the extracted and isolated compound(s) from seaweed is further modified chemically using known reactions to obtain a derivative or analog with enhanced anti-inflammatory activity compared with the unmodified compound.

Generally, the method of making the disclosed compounds includes (i) extracting a fresh seaweed specimen with an extraction solvent to produce an organic extract; (ii) subjecting the organic extract to liquid chromatography with a first mobile phase to yield a first panel of fractions, optionally (iii) subjecting one of the first panel of fractions to liquid chromatography with a second mobile phase to yield a second panel of fractions; and (iv) purifying one of the first or the second panel of fractions using HPLC with a third mobile phase to yield a compound, optionally more than one compound. Each of step (iii) and (iv) may be repeated for at least one time.

### BRIEF DESCRIPTION OF THE DRAWINGS

FIG. 1 is a schematic showing COSY and key HMBC correlations of compounds a1, a4, a5 and a8.

FIG. 2 shows key NOE correlations of compounds a1, a2, a4, a5, a6 and a8.

FIGS. 3A-3C are graphs showing the determination of concentration inducing 50% inhibition of NO production using LPS-treated RAW 264.7 and compared to Carbowax 400 0.1% v/v treated cells for compounds a1-a3 (FIG. 3A), a5, a6, a8 (FIG. 3B), and a10-a11 (FIG. 3C).

FIGS. 4A-4I are a series of graphs showing evaluation of the cytotoxic activity of compounds a1-a6, a8, a10 and a11 by measuring proliferation rate of RAW 264.7 cells. Proliferation rate was established using MTT treatment for 72 h and normalized to measurement of the initial cells plated and compared to cells treated with Carbowax 400 0.1% v/v. Statistical analysis was performed using Kruskal-Walis non-parametric test in Graphpad Prism 7.0. Graphs represent mean±SEM (* indicates P<0.05, **indicates P<0.01, ***indicates P<0.001).

### DETAILED DESCRIPTION OF THE INVENTION

### I. Definitions

As used herein, the term “heterocyclic” refers to a chain of carbon and heteroatoms, wherein the heteroatoms are selected from nitrogen, oxygen, and sulfur, at least a portion of which, including at least one heteroatom, form a ring.

As used herein, the term “amino” includes the group NH₂ (primary amino), alkylamino (secondary amino), and dialkylamino (tertiary amino), where the two alkyl groups in dialkylamino may be the same or different, i.e. alkylalkylamino. Illustratively, amino include methylamino, ethylamino, dimethylamino, methylethylamino, and the like. In addition, it is to be understood that when amino modifies or is modified by another term, such as aminoalkyl, or acylamino, the above variations of the term amino continue to apply. Illustratively, aminoalkyl includes H²N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amide” includes the group CONH₂ (primary amide), CONHalkyl (secondary amide), and CONdialkyl (tertiary amide), where the two alkyl groups in CONdialkyl may be the same or different.

As used herein, the term “prodrug” generally refers to compounds that are labile in vivo under predetermined biological conditions.

As used herein, the term “effective amount” means a dosage sufficient to prevent, treat, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

As used herein, the term “pharmaceutically acceptable” means a non-toxic material that does not interfere with the effectiveness of the biological activity of the active ingredients.

As used herein, the term “alkyl” refers to univalent groups derived from alkanes by removal of a hydrogen atom from any carbon atom. Alkanes represent saturated hydrocarbons, including those that are linear, branched, or cyclic (either monocyclic or polycyclic). An alkyl can be a linear C₁-C₃₀ alkyl, a branched C₄-C₃₀ alkyl, a cyclic C₃-C₃₀ alkyl, a linear C₁-C₃₀ alkyl or a branched C₄-C₃₀ alkyl, a linear C₁-C₃₀ alkyl or a cyclic C₃-C₃₀ alkyl, a branched C₄-C₃₀ alkyl or a cyclic C₃-C₃₀ alkyl. Optionally, alkyl groups have up to 20 carbon atoms. An alkyl can be a linear C₁-C₂₀ alkyl, a branched C₄-C₂₀ alkyl, a cyclic C₃-C₂₀ alkyl, a linear C₁-C₂₀ alkyl or a branched C₄-C₂₀ alkyl, a branched C₄-C₂₀ alkyl or a cyclic C₃-C₂₀ alkyl, a linear C₁-C₂₀ alkyl or a cyclic C₃-C₂₀ alkyl. Optionally, alkyl groups have up to 10 carbon atoms. An alkyl can be a linear C₁-C₁₀ alkyl, a branched C₄-C₁₀ alkyl, a cyclic C₃-C₁₀ alkyl, a linear C₁-C₁₀ alkyl or a branched C₄-C₁₀ alkyl, a branched C₄-C₁₀ alkyl or a cyclic C₃-C₁₀ alkyl, a linear C₁-C₁₀ alkyl or a cyclic C₃-C₁₀ alkyl. Optionally, alkyl groups have up to 6 carbon atoms. An alkyl can be a linear C₁-C₆ alkyl, a branched C₄-C₆ alkyl, a cyclic C₃-C₆ alkyl, a linear C₁-C₆ alkyl or a branched C₄-C₆ alkyl, a branched C₄-C₆ alkyl or a cyclic C₃-C₆ alkyl, or a linear C₁-C₆ alkyl or a cyclic C₃-C₆ alkyl. Optionally, alkyl groups have up to four carbons. An alkyl can be a linear C₁-C₄ alkyl, cyclic C₃-C₄ alkyl, a linear C₁-C₄ alkyl or a cyclic C₃-C₄ alkyl. Preferably, the alkyl group is unsubstituted alkyl group. Preferably, the alkyl group is a linear C₁-C₅, C₁-C₄, C₁-C₃, C₁-C₂ alkyl group, such as methyl group.

As used herein, the term “heteroalkyl” refers to alkyl groups where one or more carbon atoms are replaced with a heteroatom, such as, O, N, or S. Heteroalkyl group can be linear, branched, or cyclic. A heteroalkyl can be a linear C₁-C₃₀ heteroalkyl, a branched C₃-C₃₀ heteroalkyl, a cyclic C₂-C₃₀ heteroalkyl, a linear C₁-C₃₀ heteroalkyl or a branched C₃-C₃₀ heteroalkyl, a linear C₁-C₃₀ heteroalkyl or a cyclic C₂-C₃₀ heteroalkyl, a branched C₃-C₃₀ heteroalkyl or a cyclic C₂-C₃₀ heteroalkyl. Optionally, heteroalkyl groups have up to 20 carbon atoms. A heteroalkyl can be a linear C₁-C₂₀ heteroalkyl, a branched C₃-C₂₀ heteroalkyl, a cyclic C₂-C₂₀ heteroalkyl, a linear C₁-C₂₀ heteroalkyl or a branched C₃-C₂₀ heteroalkyl, a branched C₃-C₂₀ heteroalkyl or a cyclic C₂-C₂₀ heteroalkyl, or a linear C₁-C₂₀ heteroalkyl or a cyclic C₂-C₂₀ heteroalkyl. Optionally, heteroalkyl groups have up to 10 carbon atoms. A heteroalkyl can be a linear C₁-C₁₀ heteroalkyl, a branched C₃-C₁₀ heteroalkyl, a cyclic C₂-C₁₀ heteroalkyl, a linear C₁-C₁₀ heteroalkyl or a branched C₃-C₁₀ heteroalkyl, a branched C₃-C₁₀ heteroalkyl or a cyclic C₂-C₁₀ heteroalkyl, or a linear C₁-C₁₀ heteroalkyl or a cyclic C₂-C₁₀ heteroalkyl. Optionally, heteroalkyl groups have up to 6 carbon atoms. A heteroalkyl can be a linear C₁-C₆ heteroalkyl, a branched C₃-C₆ heteroalkyl, a cyclic C₂-C₆ heteroalkyl, a linear C₁-C₆ heteroalkyl or a branched C₃-C₆ heteroalkyl, a branched C₃-C₆ heteroalkyl or a cyclic C₂-C₆ heteroalkyl, or a linear C₁-C₆ heteroalkyl or a cyclic C₂-C₆ heteroalkyl. Optionally, heteroalkyl groups have up to four carbons. A heteroalkyl can be a linear C₁-C₄ heteroalkyl, a branched C₃-C₄ heteroalkyl, a cyclic C₂-C₄ heteroalkyl, a linear C₁-C₄ heteroalkyl or a branched C₃-C₄ heteroalkyl, a branched C₃-C₄ heteroalkyl or a cyclic C₂-C₄ heteroalkyl, or a linear C₁-C₄ heteroalkyl or a cyclic C₂-C₄ heteroalkyl.

As used herein, the term “alkenyl” refers to univalent groups derived from alkenes by removal of a hydrogen atom from any carbon atom. Alkenes are unsaturated hydrocarbons that contain at least one carbon-carbon double bond. Alkenyl group can be linear, branched, or cyclic. An alkenyl can be a linear C₂-C₃₀ alkenyl, a branched C₄-C₃₀ alkenyl, a cyclic C₃-C₃₀ alkenyl, a linear C₂-C₃₀ alkenyl or a branched C₄-C₃₀ alkenyl, a linear C₂-C₃₀ alkenyl or a cyclic C₃-C₃₀ alkenyl, a branched C₄-C₃₀ alkenyl or a cyclic C₃-C₃₀ alkenyl. Optionally, alkenyl groups have up to 20 carbon atoms. An alkenyl can be a linear C₂-C₂₀ alkenyl, a branched C₄-C₂₀ alkenyl, a cyclic C₃-C₂₀ alkenyl, a linear C₂-C₂₀ alkenyl or a branched C₄-C₂₀ alkenyl, a linear C₂-C₂₀ alkenyl or a cyclic C₃-C₂₀ alkenyl, a branched C₄-C₂₀ alkenyl or a cyclic C₃-C₂₀ alkenyl. Optionally, alkenyl groups have two to 10 carbon atoms. An alkenyl can be a linear C₂-C₁₀ alkenyl, a branched C₄-C₁₀ alkenyl, a cyclic C₃-C₁₀ alkenyl, a linear C₂-C₁₀ alkenyl or a branched C₄-C₁₀ alkenyl, a linear C₂-C₁₀ alkenyl or a cyclic C₃-C₁₀ alkenyl, a branched C₄-C₁₀ alkenyl or a cyclic C₃-C₁₀ alkenyl. Optionally, alkenyl groups have two to 6 carbon atoms. An alkenyl can be a linear C₂-C₆ alkenyl, a branched C₄-C₆ alkenyl, a cyclic C₃-C₆ alkenyl, a linear C₂-C₆ alkenyl or a branched C₄-C₆ alkenyl, a linear C₂-C₆ alkenyl or a cyclic C₃-C₆ alkenyl, a branched C₄-C₆ alkenyl or a cyclic C₃-C₆ alkenyl. Optionally, alkenyl groups have two to four carbons. An alkenyl can be a linear C₂-C₄ alkenyl, a cyclic C₃-C₄ alkenyl, a linear C₂-C₄ alkenyl or a cyclic C₃-C₄ alkenyl.

As used herein, the term “heteroalkenyl” refers to alkenyl groups in which one or more doubly bonded carbon atoms are replaced by a heteroatom. Heteroalkenyl group can be linear, branched, or cyclic. A heteroalkenyl can be a linear C₂-C₃₀ heteroalkenyl, a branched C₃-C₃₀ heteroalkenyl, a cyclic C₂-C₃₀ heteroalkenyl, a linear C₂-C₃₀ heteroalkenyl or a branched C₃-C₃₀ heteroalkenyl, a linear C₂-C₃₀ heteroalkenyl or a cyclic C₂-C₃₀ heteroalkenyl, a branched C₃-C₃₀ heteroalkenyl or a cyclic C₂-C₃₀ heteroalkenyl. Optionally, heteroalkenyl groups have up to 20 carbon atoms. A heteroalkenyl can be a linear C₂-C₂₀ heteroalkenyl, a branched C₃-C₂₀ heteroalkenyl, a cyclic C₂-C₂₀ heteroalkenyl, a linear C₂-C₂₀ heteroalkenyl or a branched C₃-C₂₀ heteroalkenyl, a linear C₂-C₂₀ heteroalkenyl or a cyclic C₂-C₂₀ heteroalkenyl, a branched C₃-C₂₀ heteroalkenyl or a cyclic C₂-C₂₀ heteroalkenyl. Optionally, heteroalkenyl groups have up to 10 carbon atoms. A heteroalkenyl can be a linear C₂-C₁₀ heteroalkenyl, a branched C₃-C₁₀ heteroalkenyl, a cyclic C₂-C₁₀ heteroalkenyl, a linear C₂-C₁₀ heteroalkenyl or a branched C₃-C₁₀ heteroalkenyl, a linear C₂-C₁₀ heteroalkenyl or a cyclic C₂-C₁₀ heteroalkenyl, a branched C₃-C₁₀ heteroalkenyl or a cyclic C₂-C₁₀ heteroalkenyl. Optionally, heteroalkenyl groups have two to 6 carbon atoms. A heteroalkenyl can be a linear C₂-C₆ heteroalkenyl, a branched C₃-C₆ heteroalkenyl, a cyclic C₂-C₆ heteroalkenyl, a linear C₂-C₆ heteroalkenyl or a branched C₃-C₆ heteroalkenyl, a linear C₂-C₆ heteroalkenyl or a cyclic C₂-C₆ heteroalkenyl, a branched C₃-C₆ heteroalkenyl or a cyclic C₂-C₆ heteroalkenyl. Optionally, heteroalkenyl groups have two to four carbons. A heteroalkenyl can be a linear C₂-C₄ heteroalkenyl, a branched C₃-C₄ heteroalkenyl, a cyclic C₂-C₄ heteroalkenyl, a linear C₂-C₄ heteroalkenyl or a branched C₃-C₄ heteroalkenyl, a linear C₂-C₄ heteroalkenyl or a cyclic C₂-C₄ heteroalkenyl, a branched C₃-C₄ heteroalkenyl or a cyclic C₂-C₄ heteroalkenyl.

As used herein, the term “alkynyl” refers to univalent groups derived from alkenes by removal of a hydrogen atom from any carbon atom. Alkynes are unsaturated hydrocarbons that contain at least one carbon-carbon triple bond. Alkynyl group can be linear, branched, or cyclic. An alkynyl can be a linear C₂-C₃₀ alkynyl, a branched C₄-C₃₀ alkynyl, a cyclic C₃-C₃₀ alkynyl, a linear C₂-C₃₀ alkynyl or a branched C₄-C₃₀ alkynyl, a linear C₂-C₃₀ alkynyl or a cyclic C₃-C₃₀ alkynyl, a branched C₄-C₃₀ alkynyl or a cyclic C₃-C₃₀ alkynyl. Optionally, alkynyl groups have up to 20 carbon atoms. An alkynyl can be a linear C₂-C₂₀ alkynyl, a branched C₄-C₂₀ alkynyl, a cyclic C₃-C₂₀ alkynyl, a linear C₂-C₂₀ alkynyl or a branched C₄-C₂₀ alkynyl, a branched C₄-C₂₀ alkynyl or a cyclic C₃-C₂₀ alkynyl. Optionally, alkynyl groups have up to 10 carbon atoms. An alkynyl can be a linear C₂-C₁₀ alkynyl, a branched C₄-C₁₀ alkynyl, a cyclic C₃-C₁₀ alkynyl, a linear C₂-C₂₀ alkynyl or a branched C₄-C₁₀ alkynyl, a branched C₄-C₂₀ alkynyl or a cyclic C₃-C₁₀ alkynyl, a linear C₂-C₂₀ alkynyl or a cyclic C₃-C₂₀ alkynyl. Optionally, alkynyl groups have up to 6 carbon atoms. An alkynyl can be a linear C₂-C₆ alkynyl, a branched C₄-C₆ alkynyl, a cyclic C₃-C₆ alkynyl, a linear C₂-C₆ alkynyl or a branched C₄-C₆ alkynyl, a branched C₄-C₆ alkynyl or a cyclic C₃-C₆ alkynyl, a linear C₂-C₆ alkynyl or a cyclic C₃-C₆ alkynyl. Optionally, alkynyl groups have up to four carbons. An alkynyl can be a linear C₂-C₄ alkynyl, a cyclic C₃-C₄ alkynyl, a linear C₂-C₄ alkynyl or a cyclic C₃-C₄ alkynyl.

As used herein, the term “heteroalkynyl” refers to alkynyl groups in which one or more triply bonded carbon atoms are replaced by a heteroatom. Heteroalkynyl group can be linear, branched, or cyclic. A heteroalkynyl can be a linear C₂-C₃₀ heteroalkynyl, a branched C₃-C₃₀ heteroalkynyl, a cyclic C₂-C₃₀ heteroalkynyl, a linear C₂-C₃₀ heteroalkynyl or a branched C₃-C₃₀ heteroalkynyl, a linear C₂-C₃₀ heteroalkynyl or a cyclic C₂-C₃₀ heteroalkynyl, a branched C₃-C₃₀ heteroalkynyl or a cyclic C₂-C₃₀ heteroalkynyl. Optionally, heteroalkynyl groups have up to 20 carbon atoms. A heteroalkynyl can be a linear C₂-C₂₀ heteroalkynyl, a branched C₃-C₂₀ heteroalkynyl, a cyclic C₂-C₂₀ heteroalkynyl, a linear C₂-C₂₀ heteroalkynyl or a branched C₃-C₂₀ heteroalkynyl, a branched C₃-C₂₀ heteroalkynyl or a cyclic C₂-C₂₀ heteroalkynyl, a linear C₂-C₂₀ heteroalkynyl or a cyclic C₂-C₂₀ heteroalkynyl. Optionally, heteroalkynyl groups have up to 10 carbon atoms. A heteroalkynyl can be a linear C₂-C₁₀ heteroalkynyl, a branched C₃-C₁₀ heteroalkynyl, a cyclic C₂-C₁₀ heteroalkynyl, a linear C₂-C₁₀ heteroalkynyl or a branched C₃-C₁₀ heteroalkynyl, a branched C₃-C₁₀ heteroalkynyl or a cyclic C₂-C₁₀ heteroalkynyl, a linear C₂-C₁₀ heteroalkynyl or a cyclic C₂-C₁₀ heteroalkynyl. Optionally, heteroalkynyl groups have two to 6 carbon atoms. A heteroalkynyl can be a linear C₂-C₆ heteroalkynyl, a branched C₃-C₆ heteroalkynyl, a cyclic C₂-C₆ heteroalkynyl, a linear C₂-C₆ heteroalkynyl or a branched C₃-C₆ heteroalkynyl, a branched C₃-C₆ heteroalkynyl or a cyclic C₂-C₆ heteroalkynyl, a linear C₂-C₆ heteroalkynyl or a cyclic C₂-C₆ heteroalkynyl. Optionally, heteroalkynyl groups have up to four carbons. A heteroalkynyl can be a linear C₂-C₄ heteroalkynyl, a branched C₃-C₄ heteroalkynyl, a cyclic C₂-C₄ heteroalkynyl, a linear C₂-C₄ heteroalkynyl or a branched C₃-C₄ heteroalkynyl, a branched C₃-C₄ heteroalkynyl or a cyclic C₂-C₄ heteroalkynyl, a linear C₂-C₄ heteroalkynyl or a cyclic C₂-C₄ heteroalkynyl.

As used herein, the term “aryl” refers to univalent groups derived from arenes by removal of a hydrogen atom from a ring atom. Arenes are monocyclic and polycyclic aromatic hydrocarbons. In polycyclic aryl groups, the rings can be attached together in a pendant manner or can be fused. Aaryl group can have six to 50 carbon atoms. An aryl can be a branched C₆-C₅₀ aryl, a monocyclic C₆-C₅₀ aryl, a polycyclic C₆-C₅₀ aryl, a branched polycyclic C₆-C₅₀ aryl, a fused poly cyclic C₆-C₅₀ aryl, or a branched fused polycyclic C₆-C₅₀ aryl. Optionally, aryl groups have six to 30 carbon atoms, i.e., C₆-C₃₀ aryl. A C₆-C₃₀ aryl can be a branched C₆-C₃₀ aryl, a monocyclic C₆-C₃₀ aryl, a polycyclic C₆-C₃₀ aryl, a branched polycyclic C₆-C₃₀ aryl, a fused polycyclic C₆-C₃₀ aryl, or a branched fused polycyclic C₆-C₃₀ aryl. Optionally, aryl groups have six to 20 carbon atoms, i.e., C₆-C₂₀ aryl. A C₆-C₂₀ aryl can be a branched C₆-C₂₀ aryl, a monocyclic C₆-C₂₀ aryl, a polycyclic C₆-C₂₀ aryl, a branched polycyclic C₆-C₂₀ aryl, a fused polycyclic C₆-C₂₀ aryl, or a branched fused polycyclic C₆-C₂₀ aryl. Optionally, aryl groups have six to twelve carbon atoms, i.e., C₆-C₁₂ aryl. A C₆-C₁₂ aryl can be a branched C₆-C₁₂ aryl, a monocyclic C₆-C₁₂ aryl, a polycyclic C₆-C₁₂ aryl, a branched polycyclic C₆-C₁₂ aryl, a fused polycyclic C₆-C₁₂ aryl, or a branched fused polycyclic C₆-C₁₂ aryl. Optionally, C₆-C₁₂ aryl groups have six to eleven carbon atoms, i.e., C₆-C₁₁ aryl. A C₆-C₁₁ aryl can be a branched C₆-C₁₁ aryl, a monocyclic C₆-C₁₁ aryl, a polycyclic C₆-C₁₁ aryl, a branched polycyclic C₆-C₁₁ aryl, a fused polycyclic C₆-C₁₁ aryl, or a branched fused polycyclic C₆-C₁₁ aryl. Optionally, C₆-C₁₂ aryl groups have six to nine carbon atoms, i.e., C₆-C₉ aryl. A C₆-C₉ aryl can be a branched C₆-C₉ aryl, a monocyclic C₆-C₉ aryl, a polycyclic C₆-C₉ aryl, a branched polycyclic C₆-C₉ aryl, a fused polycyclic C₆-C₉ aryl, or a branched fused polycyclic C₆-C₉ aryl. Optionally, C₆-C₁₂ aryl groups have six carbon atoms, i.e., C₆ aryl. A C₆ aryl can be a branched C₆ aryl or a monocyclic C₆ aryl.

As used herein, the term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). In polycyclic heteroaryl groups, the rings can be attached together in a pendant manner or can be fused. Heteroaryl group can have three to 50 carbon atoms, i.e., C₃-C₅₀ heteroaryl. A C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl. Optionally, heteroaryl groups have six to 30 carbon atoms, i.e., C₆-C₃₀ heteroaryl. A C₆-C₃₀ heteroaryl can be a branched C₆-C₃₀ heteroaryl, a monocyclic C₆-C₃₀ heteroaryl, a polycyclic C₆-C₃₀ heteroaryl, a branched polycyclic C₆-C₃₀ heteroaryl, a fused polycyclic C₆-C₃₀ heteroaryl, or a branched fused polycyclic C₆-C₃₀ heteroaryl. Optionally, heteroaryl groups have six to 20 carbon atoms, i.e., C₆-C₂₀ heteroaryl. A C₆-C₂₀ heteroaryl can be a branched C₆-C₂₀ heteroaryl, a monocyclic C₆-C₂₀ heteroaryl, a polycyclic C₆-C₂₀ heteroaryl, a branched polycyclic C₆-C₂₀ heteroaryl, a fused polycyclic C₆-C₂₀ heteroaryl, or a branched fused polycyclic C₆-C₂₀ heteroaryl. Optionally, heteroaryl groups have six to twelve carbon atoms, i.e., C₆-C₁₂ heteroaryl. A C₆-C₁₂ heteroaryl can be a branched C₆-C₁₂ heteroaryl, a monocyclic C₆-C₁₂ heteroaryl, a polycyclic C₆-C₁₂ heteroaryl, a branched polycyclic C₆-C₁₂ heteroaryl, a fused polycyclic C₆-C₁₂ heteroaryl, or a branched fused polycyclic C₆-C₁₂ heteroaryl. Optionally, C₆-C₁₂ heteroaryl groups have six to eleven carbon atoms, i.e., C₆-C₁₁ heteroaryl. A C₆-C₁₁ heteroaryl can be a branched C₆-C₁₁ heteroaryl, a monocyclic C₆-C₁₁ heteroaryl, a polycyclic C₆-C₁₁ heteroaryl, a branched polycyclic C₆-C₁₁ heteroaryl, a fused polycyclic C₆-C₁₁ heteroaryl, or a branched fused polycyclic C₆-C₁₁ heteroaryl. Optionally, C₆-C₁₂ heteroaryl groups have six to nine carbon atoms, i.e., C₆-C₉ heteroaryl. A C₆-C₉ heteroaryl can be a branched C₆-C₉ heteroaryl, a monocyclic C₆-C₉ heteroaryl, a polycyclic C₆-C₉ heteroaryl, a branched polycyclic C₆-C₉ heteroaryl, a fused polycyclic C₆-C₉ heteroaryl, or a branched fused polycyclic C₆-C₉ heteroaryl. Optionally, C₆-C₁₂ heteroaryl groups have six carbon atoms, i.e., C₆ heteroaryl. A C₆ heteroaryl can be a branched C₆ heteroaryl, a monocyclic C₆ heteroaryl, a polycyclic C₆ heteroaryl, a branched polycyclic C₆ heteroaryl, a fused polycyclic C₆ heteroaryl, or a branched fused polycyclic C₆ heteroaryl.

As used herein, the term “substituted,” means that the chemical group or moiety contains one or more substituents replacing the hydrogen atoms in the chemical group or moiety. The substituents include, but are not limited to:

a halogen atom, an alkyl group, a cycloalkyl group, a heteroalkyl group, a cycloheteroalkyl group, an alkenyl group, a heteroalkenyl group, an alkynyl group, a heteroalkynyl group, an aryl group, a heteroaryl group, a polyaryl group, a polyheteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHC₁₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{T1′})(OR^{T2′}), —OP(═O)(OR^{T1′})(OR^{T2′}), —BR^{T1′} (OR^{T2′}), —B(OR^{T1′})(OR^{T2′}), or -G′R^{T1′} in which -T′ is —O—, —S—, —NR^{T2′}—, —C(═O)—, —S(═O)—, —SO₂—, —C(═O)O—, —C(═O)NR^{T2′}—, —OC(═O) —, —NR^{T2′}C(═O)—, —OC(═O)O—, —OC(═O)NR^{T2′}—, —NR^{T2}C(═O)O—, —NR^{T2}C(═O)N R^{T3′}—, —C(═S)—, —C(═S)S—, —SC(═S)—, —SC(═S)S—, —C(═NR^{T2′})—, —C(═NR^{T2′})O—, —C(═NR^{T2′})NR^{T3′}—, —OC(═NR^{T2′})—, —NR^{T2′}C(═NR^{T3′})—, —NR^{T2′}SO₂—, —C(═NR^{T2′})NR^{T3′}—, —OC(═NR^{T2′})—, —NR^{T2′}C(═NR^{T3′})—, —NR^{T2′}SO₂—, —NR^{T2′}SO₂NR^{T3′}—, —NR^{T2′}C(═S)—, —SC(═S)NR^{T2′}—, —NR^{T2′}C(═S)S—, —NR^{T2′}C(═S)NR^{T3′}—, —SC(═NR^{T2′})—, —C(═S)NR^{T2′}—, —OC(═S)NR^{T2′}—, —NR^{T2′}C(═S)O—, —SC(═O)NR^{T2′}—, —NR^{T2′}C(═O)S—, —C(═O)S—, —SC (═O)—, —SC(═O)S—, —C(═S)O—, —OC(═S)—, —OC(═S)O—, —SO₂NR^{T2′}—, —BR^{T2′}—, or —PR^{T2′}—; where each occurrence of R^{T1′}, R^{T2′}, and R^{T3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some instances, “substituted” also refers to one or more substitutions of one or more of the carbon atoms in a carbon chain (e.g., alkyl, alkenyl, alkynyl, and aryl groups) by a heteroatom, such as, but not limited to, nitrogen, oxygen, and sulfur.

It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Use of the term “about” is intended to describe values either above or below the stated value in a range of approx. +/−10%; in other embodiments the values may range in value either above or below the stated value in a range of approx. +/−5%. The preceding ranges are intended to be made clear by context, and no further limitation is implied.

Numerical ranges disclosed in the present application of any type, disclose individually each possible number that such a range could reasonably encompass, as well as any sub-ranges and combinations of sub-ranges encompassed therein.

### II. Compounds and Compositions

C₁₅ acetogenins and their derivatives (together also referred to herein as “compounds”) having anti-inflammatory activity are disclosed herein. These compounds are suitable for use in a variety of products such as food products, cosmetics products, skin care products, nutraceuticals, and pharmaceuticals.

Formulations and compositions, such as food compositions, cosmetic formulations, skin care formulations, and pharmaceutical formulations that contain one or more of the compounds are also disclosed.

A. Compounds

The compound can contain a heterocyclic group A′ or a biheterocyclic group P′Q′. In some embodiments, the compound contains a heterocyclic group A′, where A′ is a substituted or unsubstituted five-membered heterocyclic group or a substituted or unsubstituted six-membered heterocyclic group. In some preferred embodiments, A′ of the compound is a substituted five-membered heterocyclic group or a substituted six-membered heterocyclic group, where the substituent is a halide, an hydroxyl group, a substituted or unsubstituted alkyl group, a substituted or unsubstituted alkenyl group, a substituted or unsubstituted alkynyl group, and/or an epoxide group.

In some embodiments, the compound contains a biheterocyclic group P′Q′. In some embodiments, each of P′ and Q′ is a substituted or unsubstituted heterocyclic group. In some embodiments, each of P′ and Q′ is a substituted or unsubstituted heterocyclic group. In some embodiments, each of P′ and Q′ is a substituted or unsubstituted five-membered heterocyclic group, a substituted or unsubstituted six-membered heterocyclic group, a substituted or unsubstituted seven-membered heterocyclic group, or a substituted or unsubstituted eight-membered heterocyclic group. In some embodiments, P′ of the P′Q′ moiety is a substituted or unsubstituted five-membered heterocyclic group or a substituted or unsubstituted six-membered heterocyclic group. In some embodiments, Q′ of the P′Q′ moiety is a substituted or unsubstituted five-membered heterocyclic group, a substituted or unsubstituted six-membered heterocyclic group, a substituted or unsubstituted seven-membered heterocyclic group, or a substituted or unsubstituted eight-membered heterocyclic group. In some embodiments, P′ of the P′Q′ moiety is a substituted or unsubstituted five-membered heterocyclic group and Q′ of the P′Q′ moiety is a substituted or unsubstituted eight-membered heterocyclic group. In some embodiments, P′ of the P′Q′ moiety is a substituted or unsubstituted six-membered heterocyclic group and Q′ of the P′Q′ moiety is a substituted or unsubstituted five-membered heterocyclic group, a substituted or unsubstituted six-membered heterocyclic group, or a substituted or unsubstituted eight-membered heterocyclic group. In some preferred embodiments, each of P′ and Q′ is a heterocyclic group substituted with a halide, a substituted or unsubstituted alkyl group, a substituted or unsubstituted alkenyl group, a substituted or unsubstituted alkynyl group, and/or an epoxide group.

In some embodiments, the compound has a structure of Formula (I).

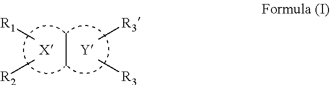

where X′ is a C₄ five-membered heterocyclic group or a C₅ six-membered heterocyclic group; Y′ is a C₄ five-membered heterocyclic group, a C₅ six-membered heterocyclic group, or a C₇ eight-membered heterocyclic group; R₁ and R₂ are independently absent, a halogen, or a substituted or unsubstituted alkyl group; R₃ and R₃′ are independently absent, a halogen, a hydroxyl group, a thiol group, an amino group,

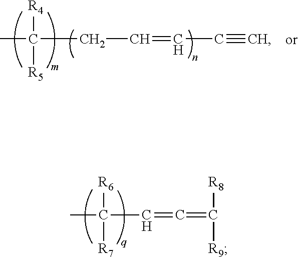

R₄-R₉ are independently a hydrogen, a halogen (e.g. a fluorine, a chlorine, a bromine, or an iodine, such as bromine or chlorine), an hydroxyl group, a thiol group, or an amino group; each of m, n, and q is an integer from 0 to 3. When R₁ and/or R₂ of Formula (I) are independently a substituted alkyl group, the substituent is a halogen (e.g. a fluorine, a chlorine, a bromine, or an iodine, such as bromine or chlorine), a hydroxyl group, a thiol group, or an amino group. In some embodiments, R₁ and R₂ of Formula (I) are independently a halogen (e.g. a fluorine, a chlorine, a bromine, or an iodine, such as bromine), a substituted or unsubstituted C₁-C₈ alkyl group, a substituted or unsubstituted C₁-C₇ alkyl group, substituted or unsubstituted C₁-C₆ alkyl group, substituted or unsubstituted C₁-C₅ alkyl group, substituted or unsubstituted C₁-C₄ alkyl group, substituted or unsubstituted C₁-C₃ alkyl group, substituted or unsubstituted C₁-C₂ alkyl group, where the substituent(s) are as defined above. In some embodiments, R₁ is absent and R₂ of Formula (I) is a halogen, a substituted or unsubstituted C₁-C₈ alkyl group, a substituted or unsubstituted C₁-C₇ alkyl group, substituted or unsubstituted C₁-C₆ alkyl group, substituted or unsubstituted C₁-C₅ alkyl group, substituted or unsubstituted C₁-C₄ alkyl group, substituted or unsubstituted C₁-C₃ alkyl group, substituted or unsubstituted C₁-C₂ alkyl group, where the substituent(s) are as defined above.

In some embodiments, the compound has a structure of Formula (II).

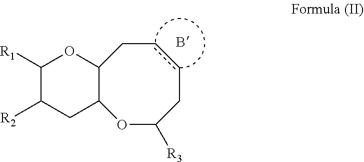

where B′ is absent or an epoxide group; R₁ and R₂ are independently a halogen or an unsubstituted alkyl group, such as an unsubstituted C₁-C₈ alkyl group, an unsubstituted C₁-C₇ alkyl group, an unsubstituted C₁-C₆ alkyl group, an unsubstituted C₁-C₅ alkyl group, an unsubstituted C₁-C₄ alkyl group, an unsubstituted C₁-C₃ alkyl group, or an unsubstituted C₁-C₂ alkyl group; R₃ is

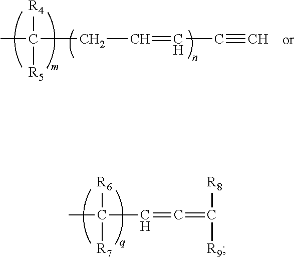

R₄-R₉ are independently a hydrogen, a halogen, an hydroxyl group, a thiol group, or an amino group; and each of m, n, and q is an integer from 0 to 3.

In some embodiments of Formula (II), R₃ is

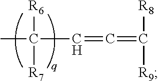

R₆-R₉ are independently a hydrogen or a halogen (e.g. fluorine, chlorine, bromine, or iodine, such as bromine); and q is an integer from 0 to 3, such as from 0 to 2, from 0 to 1, or 0.

In some embodiments, the compound has a structure of Formula (II′).

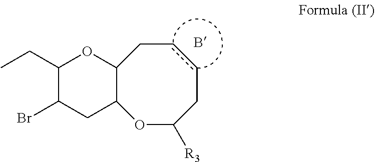

where B′ and R₃ are as defined above for Formula (II).

In some embodiments, the compound has a structure of Formula (III).

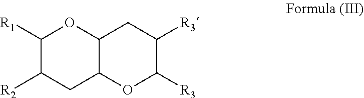

where R₁ and R₂ are independently a halogen, or an unsubstituted alkyl group, such as an unsubstituted C₁-C₈ alkyl group, an unsubstituted C₁-C₇ alkyl group, an unsubstituted C₁-C₆ alkyl group, an unsubstituted C₁-C₅ alkyl group, an unsubstituted C₁-C₄ alkyl group, an unsubstituted C₁-C₃ alkyl group, or an unsubstituted C₁-C₂ alkyl group; R₃ and R₃′ are independently a halogen,

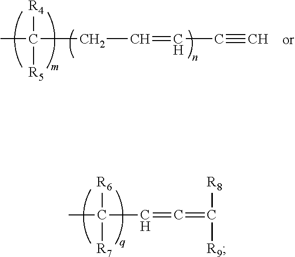

R₄-R₉ are independently a hydrogen, a halogen, an hydroxyl group, a thiol group, or an amino group; and each of m, n, and q is an integer from 0 to 3.

In some embodiments of Formula (III), R₃′ is a halogen (e.g. fluorine, chlorine, bromine, or iodine, such as bromine); R₃ is

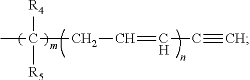

R₄ and R₅ are independently a hydrogen or a halogen; and m and n are independently an integer from 0 to 3, such as from 0 to 2, from 0 to 1, or 0.

In some embodiments, the compound has a structure of Formula (III′).

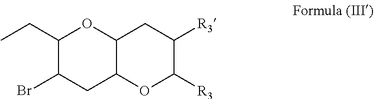

where R₃ and R₃′ are as defined above for Formula (III).

In some embodiments, the compound has a structure of Formula (IV).

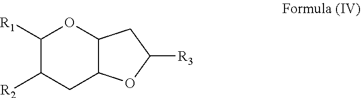

where R₁ and R₂ are independently a halogen, or an unsubstituted alkyl group, such as an unsubstituted C₁-C₈ alkyl group, an unsubstituted C₁-C₇ alkyl group, an unsubstituted C₁-C₆ alkyl group, an unsubstituted C₁-C₅ alkyl group, an unsubstituted C₁-C₄ alkyl group, an unsubstituted C₁-C₃ alkyl group, or an unsubstituted C₁-C₂ alkyl group; R₃ is

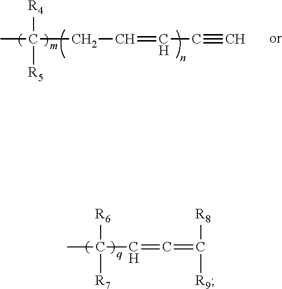

R₄-R₉ are independently a hydrogen, a halogen, a hydroxyl group, a thiol group, or an amino group; and each of m, n, and q is an integer from 0 to 3.

In some embodiments of Formula (IV), R₃ is

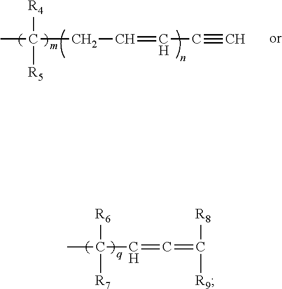

R₄-R₉ are independently a hydrogen, a halogen (e.g. fluorine, chlorine, bromine, or iodine), or a hydroxyl group; and each of m, n, and q is an integer from 0 to 3, such as from 1 to 3 or from 1 to 2.

In some embodiments, the compound has a structure of Formula (IV′).

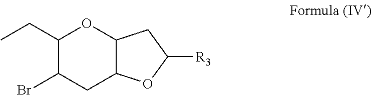

where R₃ is as defined above for Formula (IV).

In some embodiments, the compound has a structure of Formula (V).

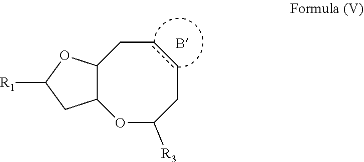

where B′ is absent or an epoxide group; R₁ is a halogen or a substituted alkyl group, such as a substituted C₁-C₈ alkyl group, a substituted C₁-C₇ alkyl group, a substituted C₁-C₆ alkyl group, a substituted C₁-C₅ alkyl group, a substituted C₁-C₄ alkyl group, a substituted C₁-C₃ alkyl group, or a substituted C₁-C₂ alkyl group, where the substituent is a halogen, a hydroxyl group, a thiol group, or an amino group; R₃ is

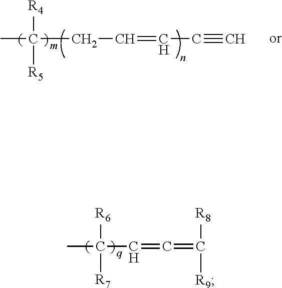

R₄-R₉ are independently a hydrogen, a halogen, a hydroxyl group, a thiol group, or an amino group; and each of m, n, and q is an integer from 0 to 3. In some embodiments of Formula (V), R₁ is a halogen substituted C₁-C₈, C₁-C₇, C₁-C₆, C₁-C₅, C₁-C₄, C₁-C₃, or C₁-C₂ alkyl group, such as a bromine substituted C₁-C₈, C₁-C₇, C₁-C₆, C₁-C₅, C₁-C₄, C₁-C₃, or C₁-C₂ alkyl group.

In some embodiments of Formula (V), R₃ is

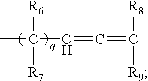

R₆-R₉ are independently a hydrogen or a halogen (e.g. fluorine, chlorine, bromine, iodine, such as bromine); and q is an integer from 0 to 3, such as from 0 to 2, from 0 to 1, or 0.

In some embodiments, the compound has a structure of Formula (V′).

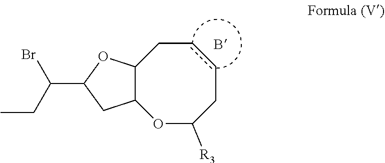

where B′ and R₃ are as defined above for Formula (V).

In some preferred embodiments, the compound has a structure of any one of a1-a8.

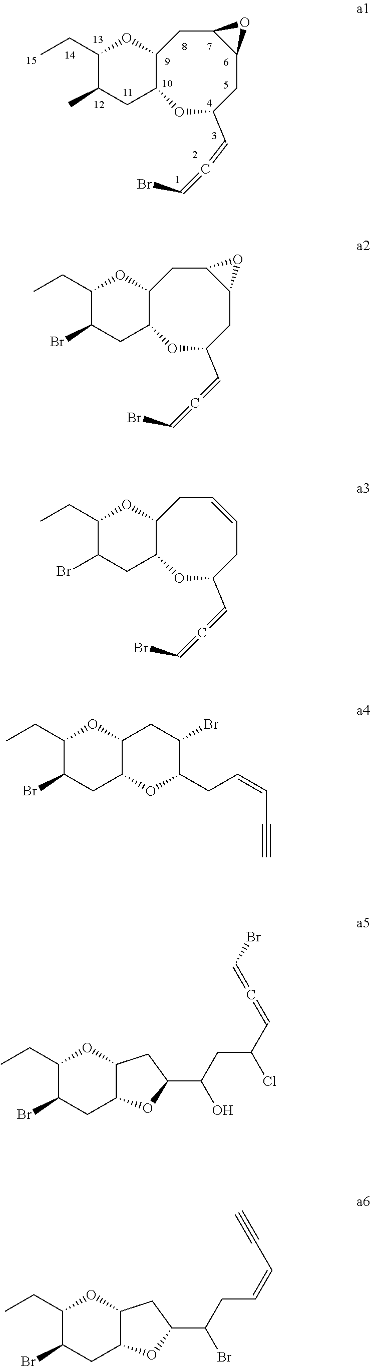

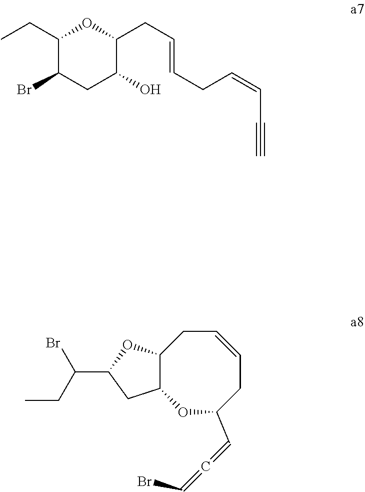

The compounds may contain one or more chiral centers or may otherwise be capable of existing as multiple stereoisomers. These may be pure (single) stereoisomers or mixtures of stereoisomers, such as enantiomers, diastereomers, and enantiomerically or diastereomerically enriched mixtures. The compounds may be capable of existing as geometric isomers. Accordingly, it is to be understood that the present invention includes pure geometric isomers or mixtures of geometric isomers.

The compounds may be neutral or may be one or more pharmaceutically acceptable salts, crystalline forms, non-crystalline forms, hydrates, or solvates, or a combination thereof. References to the compounds may refer to the neutral molecule, and/or those additional forms thereof collectively and individually from the context. Pharmaceutically acceptable salts of the compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

B. Formulations and Compositions

Typically, the compounds disclosed herein have anti-inflammatory activity with negligible toxicity, and can be used as anti-inflammatory agents in, for example, food products, cosmetics products, skin care products, nutraceuticals, and pharmaceuticals for humans, as well as in veterinary products.

Generally, depending on the route of administration, the compounds and/or the pharmaceutically acceptable salts of the compounds described herein may be formulated with a suitable excipient to form the formulation or composition. The term “excipient” is used herein to describe any ingredient in the formulation or composition other than the compounds described herein. The excipient does not decompose the compound and does not cause undesirable biological side effects or unwanted interactions in the subject to which the formulation or composition is administered. The formulations or compositions can include an effective amount of one or more compounds of any of the formulae described herein and/or their pharmaceutically acceptable salts, including any one or any combination of compounds of the formulae described herein and/or their pharmaceutically acceptable salts, for preventing, treating, or ameliorating one or more symptoms associated with inflammation in a subject. In some embodiments, the formulation or composition can further contain one or more active agents in addition to the compounds, such as other anti-inflammatory agents. Other suitable anti-inflammatory agents that can be included in the formulations are known, for example, see Erik De Clercq, Medmicro, Chapter 52 (2000).

Any one or more of the compounds provided herein can be expressly included or expressly excluded from the compositions, formulations, and/or methods of use or treatment disclosed herein.

In some embodiments, the compound itself has a physical or chemical property that is different from the physical or chemical property of the compound formulated in the formulation or composition together with a suitable excipient at an effective amount. For example, the compound by itself is a colorless oil prior to being formulated in a food composition, a cosmetic formulation, a skin care formulation, a nutraceutical formulation, or pharmaceutical formulation; the compound transforms into a different physical form, such as a liquid, an ointment, or a powder, after being formulated with an effective amount of excipient in the food composition, the cosmetic formulation, the skin care formulation, the nutraceutical formulation, or the pharmaceutical formulation. For example, the compound by itself is stable for up to a month; after being formulated with a suitable excipient at an effective amount in a food composition, a cosmetic formulation, a skin care formulation, a nutraceutical formulation, or a pharmaceutical formulation, the compound is stable for at least three months, at least 6 months, at least 1 year, at least 1.5 years, at least 2 years, up to 5 years, or up to 10 years.

1. Oral Formulations

The compounds and/or their pharmaceutically acceptable salts may be administered orally in a formulation or composition, such as a food composition, a nutraceutical formulation, or a pharmaceutical formulation. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays and liquid formulations.

Liquid formulations include suspensions, solutions, syrups, and elixirs. Such formulations may be employed as fillers in soft or hard capsules and typically comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

The compounds and/or their pharmaceutically acceptable salts may also be used in fast-dissolving, fast-disintegrating dosage forms such as those described in Expert Opinion in Therapeutic Patents, 11 (6), 981-986, by Liang and Chen (2001).

For tablet or capsule dosage forms, depending on dose, the compounds and/or their pharmaceutically acceptable salts may make up from 1 weight % to 99 weight % of the dosage form, from 1 weight % to 95 weight % of the dosage form, from 1 weight % to 90 weight % of the dosage form, from 1 weight % to 85 weight % of the dosage form, from 1 weight % to 80 weight % of the dosage form, from 1 weight % to 75 weight % of the dosage form, from 1 weight % to 70 weight % of the dosage form, more typically from 5 weight % to 60 weight % of the dosage form, from 1 weight % to 50 weight % of the dosage form, from 1 weight % to 20 weight % of the dosage form, or from 1 weight % to 10 weight % of the dosage form. In addition to the compounds described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets or capsules may also optionally contain surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets or capsules also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include glidants (e.g. Talc or colloidal anhydrous silica at about 0.1 weight % to about 3 weight %), anti-oxidants, colourants, flavouring agents, preservatives and taste-masking agents.

Exemplary tablets contain up to about 80% of one or more of the compounds described herein, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet or capsule blends may be compressed directly or by roller to form tablets. Tablet or capsule blends or portions of blends may alternatively be wet-, dry-, or melt-granulated, melt congealed, or extruded before tableting. The final formulation may contain one or more layers and may be coated or uncoated; it may even be encapsulated.

Solid formulations for oral administration may be formulated to be immediate and/or modified release. Modified release formulations include delayed, sustained, pulsed, controlled, targeted and programmed release formulations.

2. Parenteral Formulations

The compounds and/or their pharmaceutically acceptable salts may also be administered directly into the blood stream, into muscle, or into an internal organ in a pharmaceutical formulation. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

Parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, using a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof. Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine.

The formulation can contain a preservative to prevent the growth of microorganisms. A preservative is a substance that prevents or inhibits microbial growth and extends the shelf life of the drug products. Suitable preservatives include, but are not limited to, parabens, chlorobutanol, phenol, sodium benzoate, EDTA and sorbic acid, and thimerosal. For a liquid or semi-solid pharmaceutical dosage form, it is crucial to include a preservative in the formulation. Commonly used preservatives in these systems include sodium benzoate, EDTA, sorbic acid, and parabens. The formulation may also contain an antioxidant to prevent degradation of the active agent(s).

Preservative concentrations recommended for parental preparation

| | |
|---|---|
| Benzyl Alcohol | 0.5 to 10% |
| Benzalkonium Chloride | 0.01% |
| Butyl Paraben | 0.015% |
| Chlorobutanol | 0.25 to 0.5% |
| Meta Cresol | 01 to 0.25% |
| Chlorocresol | 0.1 to 0.18% |
| Methyl Paraben | 0.01 to 0.5% |
| Phenyl Ethyl Alcohol | 0.25 to 0.002% |
| Propyl Paraben | 0.005 to 0.002% |
| Phenol | 0.065 to 0.02% |

Preservative Concentration for Liquid Oral Preparation

| | |
|---|---|
| Benzonic Acid | 0.1 to 0.2% |
| Sorbic Acid | 0.1 to 0.2% |
| Methyl Paraben | 0.25% |
| Propyl Paraben | 0.5 to 0.25% |
| Sodium Benzonate | 0.1 to 0.2% |
| Bronidol | 0.001 to 0.05% |
| Propylene Glycol | 0.25% |

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

The preparation of parenteral formulations under sterile conditions, for example, by lyophilisation, may readily be accomplished using standard pharmaceutical techniques well known to those skilled in the art.

The solubility of the compounds used in the preparation of a parenteral formulation may be increased by the use of appropriate formulation techniques, such as the incorporation of solubility-enhancing agents.

Formulations for parenteral administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

(a) Controlled Release Formulations

The parenteral formulations described herein can be formulated for controlled release including immediate release, delayed release, extended release, pulsatile release, and combinations thereof.

1. Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In embodiments wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials, which are insoluble in aqueous solution or slowly soluble in aqueous solution but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

2. Method of making Nano- and Microparticles

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water-soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

(b) Injectable/Implantable Formulations

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

3. Enteral Formulations

Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

(a) Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

(1) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename EUDRAGIT t®. In further preferred embodiments, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

(2) Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

4. Pulmonary and Mucosal Formulations

The compounds and/or their pharmaceutically acceptable salts can be formulated for pulmonary or mucosal administration in a pharmaceutical formulation. The administration can include delivery of the composition to the lungs, nasal, oral (sublingual, buccal), vaginal, or rectal mucosa.

For example, the compounds can also be administered intranasally or by oral inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may comprise a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebuliser contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compounds described herein, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation for use in an atomizer using electrohydrodynamics to produce a fine mist may contain from 1 μg to 20 mg of one or more of the compounds per actuation and the actuation volume may vary from 1 μl to 100 μl. A typical formulation may contain one or more of the compounds described herein, propylene glycol, sterile water, ethanol and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff”. The overall daily dose will be administered in a single dose or, more usually, as divided doses throughout the day.

In some embodiments, the compounds and/or their pharmaceutically acceptable salts can be formulated for pulmonary delivery, such as intranasal administration or oral inhalation. Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. A suitable saline content and pH for an innocuous aqueous solution for nasal and/or upper respiratory administration can be readily determined by a person skilled in the art.

In some embodiments, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some embodiments, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some embodiments, the pharmaceutical formulations may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of compounds by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+ nebulizer (PARI Respiratory Equipment, Monterey, Calif.).

5. Topical Formulations

The compounds and/or their pharmaceutically acceptable salts may be administered topically to a subject in need thereof in a pharmaceutical formulation, a cosmetic formulation, or a skin care formulation. The compounds and/or their pharmaceutically acceptable salts may be administered directly to the external surface of the skin or the mucous membranes (including the surface membranes of the nose, lungs and mouth), such that the compounds and/or their pharmaceutically acceptable salts cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

In some embodiments, the dermatologically acceptable carriers include hydro-alcoholic systems and oil-in-water emulsions. When the dermatologically acceptable carrier is a hydro-alcoholic system, the carrier can contain from about 0% to about 99% of ethanol, isopropanol, or mixtures thereof, and from about 1% to about 99% of water. More preferred is a carrier containing from about 5% to about 60% of ethanol, isopropanol, or mixtures thereof, and from about 40% to about 95% of water. Especially preferred is a carrier containing from about 20% to about 50% of ethanol, isopropanol, or mixtures thereof, and from about 50% to about 80% of water. When the carrier is an oil-in-water emulsion, the carrier can include any of the common excipient ingredients for preparing these emulsions. A more detailed discussion of suitable carriers is found in U.S. Pat. No. 5,605,894 to Blank et al., and, U.S. Pat. No. 5,681,852 to Bissett, both of which are herein incorporated by reference in their entirety.

When the compounds and/or their pharmaceutically acceptable salts are formulated in a cosmetic or skin care formulation, additional cosmetic agents may be included. Non-limiting examples of such cosmetic agents include vitamin B3 compounds such as those described in WO 97/39733 by Oblong et al., herein incorporated by reference in its entirety; hydroxy acids such as salicylic acid; exfoliation or desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; other anti-inflammatory agents; anti-oxidants/radical scavengers such as tocopherol and esters thereof; metal chelators, especially iron chelators; retinoids such as retinol, retinyl palmitate, retinyl acetate, retinyl propionate, and retinal; N-acetyl-L-cysteine and derivatives thereof; hydroxy acids such as glycolic acid; keto acids such as pyruvic acid; benzofuran derivatives; depilatory agents (e.g., sulfhydryl compounds); skin lightening agents (e.g., arbutin, kojic acid, hydroquinone, ascorbic acid and derivatives such as ascorbyl phosphate salts, placental extract, and the like); anti-cellulite agents (e.g., caffeine, theophylline); moisturizing agents; anti-microbial agents; anti-androgens; and skin protectants. Mixtures of any of the above mentioned cosmetic agents may also be used. A more detailed description of these cosmetic agents is found in U.S. Pat. No. 5,605,894 to Blank et al.

Other conventional skin care additives may also be included in the cosmetic or skin care formulation. For example, urea, guanidine, glycerol, petrolatum, mineral oil, sugar esters and polyesters, polyolefins, methyl isostearate, ethyl isostearate, cetyl ricinoleate, isononyl isononanoate, isohexadecane, lanolin, lanolin esters, cholesterol, pyrrolidone carboxylic acid/salt (PCA), trimethyl glycine (betaine), tranexamic acid, amino acids (e.g., serine, alanine, threonine, histidine), their salts, panthenol and its derivatives, collagen, hyaluronic acid, elastin, hydrolysates, primrose oil, jojoba oil, epidermal growth factor, soybean saponins, mucopolysaccharides, and mixtures thereof may be used. Other suitable skin care additives are discussed in WO 97/39733 by Oblong et al.

Formulations for topical administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

6. Effective Doses

Effective doses of the present compounds depend on many factors, including the indication being treated, the route of administration, co-administration of other therapeutic compositions, and the overall condition of the subject.

In general, treatment regimens utilizing compounds comprise administration of from about 0.01 mg to about 300 mg of the compounds per kilogram body weight of the recipient per day in multiple doses or in a single dose. In some embodiments, a suitable dose may be in the range of 0.1 to 300 mg per kilogram body weight of the recipient per day, optionally in the range of 0.5 to 300 mg per kilogram body weight of the recipient per day, in the range of 1 to 300 mg per kilogram body weight of the recipient per day, in the range of 2 to 300 mg per kilogram body weight of the recipient per day, in the range of 0.01 to 150 mg per kilogram body weight of the recipient per day, in the range of 0.01 to 100 mg per kilogram body weight of the recipient per day, in the range of 0.1 to 150 mg per kilogram body weight of the recipient per day, in the range of 0.1 to 100 mg per kilogram body weight of the recipient per day, in the range of 1 to 150 mg per kilogram body weight of the recipient per day, in the range of 1 to 100 mg per kilogram body weight of the recipient per day, in the range of 5 to 150 mg per kilogram body weight per day, in the range of 5 to 100 mg per kilogram body weight of the recipient per day, in the range of 10 to 100 mg per kilogram body weight of the recipient per day, and optionally in the range 15 to 100 mg per kilogram body weight per day.

The desired dose may be presented as two, three, four, five or six or more sub-doses administered at appropriate intervals throughout the day. These sub-doses may be administered in unit dosage forms, for example, containing from 0.1 to 1500 mg, from 0.5 to 1500 mg, from 1 to 1500 mg, from 5 to 1500 mg, from 10 to 1500 mg, 0.1 to 1000 mg, from 0.5 to 1000 mg, from 1 to 1000 mg, from 5 to 1000 mg, from 10 to 1000 mg, from 20 to 1000 mg, from 50 to 1000 mg, or from 50 to 700 mg of the compounds per unit dosage form.

### III. Methods of Making

Methods of making the compounds are disclosed herein. An exemplary method of making the compounds is extracting and isolating them from seaweed, for example, Laurencia sp., such as a Saudi Arabian Red Sea Laurencia sp. In some embodiments, the extracted and isolated compound from seaweed is further modified chemically using known reactions to obtain a derivative or analog with enhanced anti-inflammatory activity compared with the unmodified compound.

A. Extracting and Isolating Compounds from Seaweed

Generally, the method includes (i) extracting a fresh seaweed specimen with an extraction solvent to produce an organic extract; (ii) subjecting the organic extract to liquid chromatography with a first mobile phase to yield a first panel of fractions, optionally (iii) subjecting one of the first panel of fractions to liquid chromatography with a second mobile phase to yield a second panel of fractions; and (iv) purifying one of the first or the second panel of fractions using HPLC with a third mobile phase to yield a compound, optionally more than one compound.

When step (iii) is performed, it may be repeated for at least one time, at least two times, at least three times, at least five times, at least 10 times, or up to 20 times. Each repeat of step (iii) may be performed prior to, simultaneously with, or subsequent to step (iv). Each repeat of step (iii) may be performed to separate the same fraction of the first panel of fractions or a different fraction of the first panel of fractions from the previous liquid chromatography separation, and may use the same mobile phase or a different mobile phase from the previous liquid chromatography separation.

Optionally, step (iv) is repeated for at least one time, at least two times, at least three times, at least five times, at least 10 times, or up to 20 times. Each repeat of step (iv) may be performed to purify the same fraction of the first panel of fractions or second panel of fractions or a different fraction of the first panel of fractions or second panel of fractions from the previous purification and may use the same mobile phase or a different mobile phase from the previous purification.

An exemplary method is described in Example 1 below. This is a cost-effective way to produce significant quantities of the disclosed compounds, which provides an advantage in the production of cosmetics and nutraceuticals compared to existing products. Large scale open-sea aqua-farming-based biosynthesis of the compounds can be performed.

1. Extracting a Fresh Seaweed Specimen

Generally, prior to extraction, a seaweed specimen is collected at a site. For example, a seaweed specimen of Laurencia sp. is collected from the Red Sea at different coastal locations or cultivated in indoor aquaria. The collected seaweed specimen is stored at a temperature in a range from 2° C. to 15 t to keep it fresh until further processing.

The fresh seaweed specimen can be extracted with a suitable extraction solvent to produce an organic extract. Suitable extraction solvents include, but are not limited to, dichloromethane, methanol, ethanol, chloroform, acetone, and hexane, and a mixture thereof. For example, the fresh seaweed specimen is extracted with a mixture of dichloromethane and methanol (1:1, v/v) to produce the organic extract. Typically, the extraction step is performed at room temperature (i.e. 20-22° C. or 68-72° F.) under 1 atm.

Optionally, the solvent in the organic extract of the seaweed specimen is evaporated in air or vacuo prior to the separation process.

2. Subjecting the Extract to Liquid Chromatography

Generally, the organic extract or the organic extract after solvent evaporation produced from step (i) is subjected to liquid chromatography using a mobile phase to separate the extract into a panel of fractions.

Liquid chromatography is a known separation technique which is carried out by passing the sample using a suitable mobile phase through a column or a plane. Exemplary solvents for the mobile phase include, but are not limited to, acetonitrile, water, methanol, ethanol, hexane (cHex and nHex), n-heptane, dichloromethane, dichloroethane, diethyl ether, methyl acetate, ethyl acetate (EtOAc), acetone, and isopropanal, and a mixture thereof. When a mixture of two or more solvents are used in the mobile phase, the volume ratio of the solvents may be adjusted to produce a mobile phase having increasing or decreasing polarity. The mobile phase may contain two separate solvents where one of the solvents is first applied and the second solvent is subsequently applied. For example, an extract is separated by liquid chromatography using a first solvent which is a mixture of cHex/EtOAc and subsequently a second solvent which is methanol.

Columns and planes for carrying out liquid chromatography are commercially available, such as silica gel column and C₁₈ column.

For example, the organic extract or the organic extract after solvent evaporation produced from step (i) is subjected to liquid chromatography over silica gel using a mixture of hexane and ethyl acetate of increasing polarity and then methanol, to separate the extract into a first panel of fractions, which contains at least two fractions, at least three fractions, at least four fractions, at least five fractions, at least six fractions, at least seven fractions, at least eight fractions, at least nine fractions, at least ten fractions, or up to twenty fractions, such as seven fractions (i.e. fractions A-G).

Typically, one of the fractions is subjected to liquid chromatography with the same or a different mobile phase from the previous separation for further separation of this fraction. For example, one of the fractions A-G, such as fraction A, is subjected to liquid chromatography over silica gel using a mixture of hexane and ethyl acetate of increasing polarity to further separate fraction A into a second panel of fractions, which contains at least two fractions, at least three fractions, at least four fractions, at least five fractions, at least six fractions, at least seven fractions, at least eight fractions, at least nine fractions, at least ten fractions, or up to twenty fractions, such as seven fractions (i.e. fractions A1-A7). This further liquid chromatography separation step may be performed more than one time and each time it is performed to further separate any one of the fractions in the first panel of fractions.

In some embodiments, one of the first panel of fractions or one of the second panel of fractions contains a compound in pure form.

3. Purifying a Fraction Using HPLC

Generally, one of the first panel of fractions or one of the second panel of fractions is purified using HPLC with a suitable mobile phase to yield a compound, optionally more than one compound, such as two compounds, three compounds, or four compounds.

Any of the exemplary mobile phases for carrying out liquid chromatograph may be used in HPLC for purification of the fractions. For example, a mixture of hexanes and ethyl acetate of different polarity, such as cHex/EtOAc (98:2, v/v), cHex/EtOAc (95:5, v/v), cHex/EtOAc (94:6, v/v), cHex/EtOAc (83:17, v/v). Two or more solvents may be consecutively applied for purification of the fractions. For example, a fraction is purified by HPLC using a mixture of cHex/EtOAc (95:5, v/v) and subsequently nHex/EtOAc (94:6, v/v). For example, a fraction is purified by HPLC using a mixture of cHex/EtOAc (83:17) and subsequently cHex/acetone (95:15).

This HPLC purification step (i.e. step (iv)) may be repeated at least one time, at least two times, at least three times, at least five times, at least 10 times, or up to 20 times. Each repeat of step (iv) may be performed to purify the same fraction or a different fraction of the first panel of fractions or the same fraction or a different fraction of the second panel of fractions from the previous purification. Each repeat of step (iv) may use the same mobile phase or a different mobile phase from the previous purification.

For example, fraction A2 of the second panel of fractions A1-A7 above is repeatedly purified by a normal-phase HPLC using cHex/EtOAc (98:2) as the mobile phase to yield compound a3, compound a4, compound a6, and compound a8; and fraction A6 of the second panel of fractions A1-A7 is purified by a normal-phase HPLC using cHex/EtOAc (83:17) as the mobile phase to yield compound a7. For example, fraction B of the first panel of fractions A-G above is purified by a normal-phase HPLC using cHex/EtOAc (83:17) and subsequently cHex/acetone (95:15) as the mobile phase to yield compound a1, compound a2, compound a5, and compound a7.

In some embodiments, the method yields other metabolites that have anti-inflammatory activities in addition to the disclosed compounds, such as metabolites a9-a11 shown below.

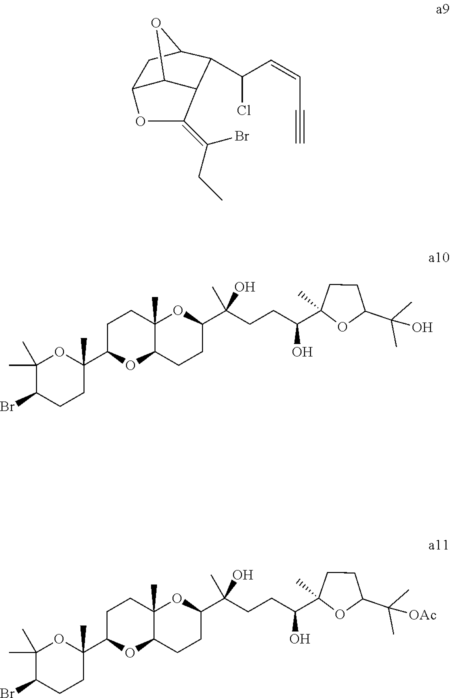 [SMILES: C#C/C=C\C(Cl)C1C2CC3O/C(=C(/Br)CC)C1C3O2 ; CC(C)(O)C1CC[C@](C)([C@@H](O)CC[C@](C)(O)[C@H]2CC[C@H]3O[C@@H]([C@]4(C)CC[C@@H](Br)C(C)(C)O4)CC[C@]3(C)O2)O1 ; CC(=O)OC(C)(C)C1CC[C@](C)([C@@H](O)CC[C@](C)(O)[C@H]2CC[C@H]3O[C@@H]([C@]4(C)CC[C@@H](Br)C(C)(C)O4)CC[C@]3(C)O2)O1]

For example, fraction A5 of the second panel of fractions A1-A7 is purified by a normal-phase HPLC using cHex/EtOAc (95:5) and subsequently nHex/EtOAc (94:6) as the mobile phase to yield metabolite a9; metabolite a10 and metabolite a11 are produced in pure form by liquid chromatography.

4. Optional Steps

The method may include one or more of: (a) establishing a robust culture protocol to grow Laurencia sp. in aquaculture, developing a scalable prototype culture system for open-sea farm applications; (b) improving Laurencia sp. harvest and culture conditions to increase the yield and desired profile of compounds of interest (varying the temperature, UV radiation, photoperiod, chemical clues of herbivores); (c) optimizing chemical extraction protocol for the compounds of interest and/or large-scale isolation of the compounds; (d) modifying the chemical structure of the targeted compounds to enhance their activity, and (e) testing the activity of the compounds, in tissue cultures and in vivo model animals, to further ensure lack of toxicity or other adverse effects in products, such as nutraceuticals or skin care products.

A system can be designed to identify the best growth conditions (hook to a substrate, running seawater, addition of selected nutrients). Tests can be performed to identify temperature growth responses, optimum, photoperiod responses. Experiments can be conducted with doses of UVB radiation and the presence of predators, in an effort to induce physiological and molecular responses for photo-protection, reduce oxidative stress and reparatory systems, as well as chemical defense to predators. These experiments aim at improving the desired profile of enriched chemical compounds of interest.

To determine chemical identity, stability and purity of isolated compounds, the already used and successful state of the art analytical and spectroscopic techniques can be utilized. The upscaling of the isolation can be performed and resulting routines selected to achieve the best amounts of desired compounds at the lowest costs to increase even further the commercialization potential. The chemical modifications, derivatisation of the existing compounds and subsequent evaluation of the properties of those derived analogues can be carried out.

### IV. Methods of Using

Methods of using the compounds as anti-inflammatory agents for preventing, treating, or ameliorating one or more symptoms associated with an inflammation in a subject are disclosed.

Generally, the method includes (i) administering to the subject an effective amount of the compound(s) to prevent, treat, or ameliorate one or more symptoms associated with inflammation in the subject. The subject can be a mammal. The compound(s) can be administered by a medical professional or the subject being treated (e.g. self-administration).

In some embodiments of the method, an effective amount of metabolites extracted and isolated from a seaweed, which includes one or more compounds disclosed herein and optionally one or more metabolites having anti-inflammatory activity in addition to the compound(s), is administered to the subject to prevent, treat, or ameliorate one or more symptoms associated with inflammation in the subject. For example, an effective amount of metabolites extracted and isolated from Laurencia sp., which includes one or more compounds disclosed herein and one or more metabolites selected from a9-a11, is administered to the subject to prevent, treat, or ameliorate one or more symptoms associated with inflammation in the subject. Examples of symptoms associated with acute inflammation, include, but are not limited to pain, redness, loss ff of function, swelling and heat.

Common inflammations are known, for example, as described in “everything you need to know about inflammation” retrieved from https://www.medicalnewstoday.com/articles/248423; Miyasaka and Takatsu, “Chronic Inflammation: Mechanism and Regulation”, Springer, November 2016; Chatterjee, et al., “Immunity and Inflammation in Health and Disease”, 1^{st} Edition, Academic Press, September 2017.

In some embodiments, the compound(s) administered to the subject is in an effective amount to inhibit nitric oxide (“NO”) production in the subject. Nitric oxide (NO) is a signaling molecule that plays a key role in the pathogenesis of inflammation. It gives an anti-inflammatory effect under normal physiological conditions. On the other hand, NO is considered as a pro-inflammatory mediator that induces inflammation due to over production in abnormal situations. NO is synthesized and released into the endothelial cells by the help of NOSs that convert arginine into citrulline producing NO in the process. Oxygen and NADPH are necessary co-factors in such conversion. NO is believed to induce vasodilatation in cardiovascular system and furthermore, it involves in immune responses by cytokine-activated macrophages, which release NO in high concentrations. In addition, NO is a potent neurotransmitter at the neuron synapses and contributes to the regulation of apoptosis. NO is involved in the pathogenesis of inflammatory disorders of the joint, gut and lungs. Therefore, NO inhibitors represent important therapeutic advance in the management of inflammatory diseases Inflammatory conditions that can be treated using the compositions disclosed herein include, but are not limited to inflammatory bowel disease, ulcerative colitis, conditions in which excess production of nitric oxide is implicated. Excessive nitric oxide production is recognized in septic shock and cardiogenic shock, post-traumatic immunodepression, psoriasis, atopic dermatitis, irritant dermatitis, allergic dermatitis, lupus erythematous, sunburn-induced flushing, nerve-mediated flushing and skin swelling. Trauma may be surgical or non-surgical (e.g., accidental). Post-traumatic immunodepression can result from a trauma, which may be an incision, laceration, tear, burn, or crushing injury of a tissue, where the tissue may be skin, non-cardiac muscle, bone, and/or an internal organ such as but not limited to the liver, lung, spleen, heart, gastrointestinal tract, or brain.

The NO may be produced in macrophage cells in the subject. For example, the compound(s) administered to the subject has an IC₅₀ for NO production inhibition below about 40 μM, below about 35 μM, below about 30 μM, below about 28 μM, below about 25 μM, below about 20 μM, below about 15 μM, below about 10 μM, or below about 5 μM, against macrophage cells.

In some embodiments, the amount of compound(s) administered to the subject is effective to inhibit NO production with no cytotoxicity. For example, the compound(s) administered to the subject has an IC₅₀ for NO production inhibition below about 30 μM and a cytotoxicity at a concentration higher than 30 μM, an IC₅₀ for NO production inhibition below about 20 μM and a cytotoxicity at a concentration higher than about 30 μM, an IC₅₀ for NO production inhibition below about 15 μM and a cytotoxicity at a concentration higher than about 30 μM, an IC₅₀ for NO production inhibition below about 5 μM and a cytotoxicity at a concentration higher than about 15 μM, an IC₅₀ for NO production inhibition below about 15 μM and a cytotoxicity at a concentration higher than about 15 μM, an IC₅₀ for NO production inhibition below about 5 μM and a cytotoxicity at a concentration higher than about 5 μM, or an IC₅₀ for NO production inhibition below about 5 μM and a cytotoxicity at a concentration higher than about 7 μM, against macrophage cells.

In some embodiments, the compounds and/or their pharmaceutically acceptable salts can be administered in the form of a pharmaceutical composition or formulation in association with one or more pharmaceutically acceptable excipients, such as the pharmaceutical composition or formulation described above. The choice of the pharmaceutically acceptable excipients will to a large extent depend on factors such as the particular mode of administration, the effect of the excipient on solubility and stability, and the nature of the dosage form.

1. Administration Routes

The compound(s) and/or their pharmaceutically acceptable salts or the composition or formulation containing the compound(s) and/or their pharmaceutically acceptable salts can be administered to the subject by oral administration, parenteral administration, inhalation, mucosal administration, or topical administration, or a combination thereof.

For example, the compound(s) and/or their pharmaceutically acceptable salts or the composition or formulation containing the compound(s) and/or their pharmaceutical acceptable slats can be orally administered to a subject by a medical professional or the subject being treated (e.g. self-administration). The compound(s) or the composition or formulation containing the compound(s) and/or their pharmaceutical acceptable slats can be administered as tablets, capsules containing particulates, granules, powders, lozenges (including liquid-filled lozenges), chews, multi- and nano-particulates, gels, or liquids (e.g. solution or suspensions in aqueous or non-aqueous solvent).

Optionally, the compound(s) and/or their pharmaceutically acceptable salts or the composition or formulation containing the compound(s) and/or their pharmaceutical acceptable slats can be administered to the subject by intravenous injection or intraperitoneal injection. The intravenous injection or intraperitoneal injection can be performed by a medical professional or the subject being treated (e.g. self-injection).

Alternatively, the compound(s) and/or their pharmaceutically acceptable salts or the composition or formulation containing the compound(s) and/or their pharmaceutical acceptable slats can be administered to the subject by inhalation, such as mouth inhalation and/or nasal inhalation.

Optionally, the compound(s) and/or their pharmaceutically acceptable salts or the composition or formulation containing the compound(s) and/or their pharmaceutical acceptable slats can be administered to the subject by topically applying the compound(s) or the pharmaceutical composition or formulation on one or more of the exposed surfaces of the subject.

2. Optional Steps

a. Administering Additional Active Agent(s)

One or more active agents in addition to the compounds may be administered to the subject throughout the method or at different intervals during the method. For example, the one or more additional active agents is administered to the subject prior to, during, and/or subsequent to step (i).

In some embodiments, the one or more additional active agents is included in the composition or formulation containing the compound(s) and is administered to the subject simultaneously with the compound(s) in the composition or formulation in association with one or more pharmaceutically acceptable excipients. For example, the one or more additional active agents is one or more metabolites extracted and isolated from a seaweed, such as metabolites a9-a11 extracted and isolated from Laurencia sp., and is formulated into a composition or formulation together with the compound(s); the composition or formulation is administered to the subject.

In some embodiments, the one or more additional active agents are one or more known anti-inflammatory agents, such as those described in Maroon, et al., “Natural anti-inflammatory agents for pain relief”, Surg. Neurol. Int., 1:80 (2010); and “nonsteroidal anti-inflammatory drugs” on https://www.drugs.com/drug-class/nonsteroidal-anti-inflammatory-agents.html. The amount of the one or more anti-inflammatory agents required will vary from subject to subject according to their need.

The present invention will be further understood by reference to the following non-limiting examples.

### EXAMPLES

### 1. Materials and Methods

### 1.1. General Experimental Procedures

Optical rotations were measured on a Krüss polarimeter with a 1 dm cell. UV spectra were recorded on a Perkin Elmer Lambda 40 UV/Vis spectrophotometer. IR spectra were obtained on a FTIR Bruker Alpha II spectrometer. High-resolution APCI mass spectra were measured on a Thermo Scientific LTQ Orbitrap Velos mass spectrometer (Institute of Biology, Medicinal Chemistry and Biotechnology, National Hellenic Research Foundation). NMR spectra were recorded on Bruker Avance NEO 950, Bruker Avance NEO 700, Bruker Avance III 600, and Bruker DRX 400 spectrometers. Chemical shifts are given on a δ (ppm) scale using TMS as internal standard.

The 2D experiments (HSQC, HMBC, COSY, NOESY) were performed using standard Bruker pulse sequences. Column chromatography separations were performed with Kieselgel 60 (Merck). HPLC separations were conducted using a Waters 600 liquid chromatography pump equipped with a Waters 410 differential refractometer, using the column Econosphere Silica 10 u (Alltech, 25 cm×10 mm). TLC were performed with Kieselgel 60 F254 (Merck aluminum support plates) and spots were detected after spraying with 20% H₂SO₄ in MeOH reagent and heating at 100° C. for 1 min.

### 1.2. Biological Material

Specimens of Laurencia sp. were collected by hand from Rose Reef (GPS coordinates 22° 18′ N, 38° 53′ E) out off of the village of Thuwal in the Red Sea coast of the Kingdom of Saudi Arabia, at a depth of 1.5-2 m in January 2018. A voucher specimen of the alga has been deposited at the Herbarium of the Section of Pharmacognosy and Chemistry of Natural Products, Department of Pharmacy, National and Kapodistrian University of Athens (ATPH/MP0677).

### 1.3. Extraction and Isolation

The algal specimens were exhaustively extracted with mixtures of CH2Cl2/MeOH at room temperature. After evaporation of the solvent in vacuo, the organic extract (288.0 mg) was subjected to vacuum liquid chromatography over silica gel using cHex with increasing amounts of EtOAc and finally MeOH as the mobile phase to yield 7 fractions (A-G), among which 10 (11.7 mg) and 11 (8.0 mg) were isolated in pure form. Fraction A (102.8 mg) was subjected to vacuum liquid chromatography over silica gel using mixtures of nHex and EtOAc of increasing polarity as eluent to afford 7 fractions (A1-A7). Fraction A2 (37.9 mg) was repeatedly purified by normal phase HPLC using cHex/EtOAc (98:2) as the mobile phase to afford 3 (2.9 mg), 4 (0.5 mg), 6 (4.1 mg) and 7 (1.0 mg). Fraction A5 (6.7 mg) was purified by normal phase HPLC using cHex/EtOAc (95:5) and nHex/EtOAc (94:6) to yield 9 (1.5 mg). Fraction A6 (4.0 mg) was subjected to further fractionation by normal phase HPLC using cHex/EtOAc (83:17) as the mobile phase to yield 8 (2.0 mg). Fraction B (65.6 mg) was subjected to normal phase HPLC using cHex/EtOAc (83:17) and cHex/acetone (95:15) as mobile phase to afford 1 (33.0 mg), 2 (3.3 mg), 5 (3.8 mg) and 8 (2.2 mg).

Thuwalallene A (a1): Colorless oil; [α]20D −52 (c 2.81, CHCl₃); UV (CHCl3) λmax (log ε) 241 (3.30); IR (thin film) νmax 2961, 2925, 2880, 2855, 1723, 1480, 1439, 1090, 660 cm-1; 1H and 13C NMR data, see Tables 1 and 2; HR-APCIMS m/z 406.9839, 408.9818, 410.9797 [M+H]+ (54:100:48) (calcd. for C15H2179Br2O3, 406.9852, C15H2179Br81BrO3, 408.9831, C15H2181Br2O3, 410.9811).

Thuwalallene B (a2): Colorless oil; [α]20D −77 (c 0.33, CHCl₃); UV (CHCl₃) λmax (log ε) 242 (2.98); IR (thin film) νmax 2961, 2927, 2876, 2853, 1713, 1452, 1382, 1085, 779, 662 cm-1; 1H and 13C NMR data, see Tables 1 and 2; HR-APCIMS m/z 406.9846, 408.9825, 410.9831 [M+H]+ (54:100:50) (calcd. for C15H2179Br2O3, 406.9852, C15H2179Br81BrO3, 408.9831, C15H2181Br2O3, 410.9811).

Thuwalallene C (a3): Colorless oil; [α]20D −300 (c 0.01, CHCl₃); UV (CHCl₃) λmax (log ε) 242 (2.97); IR (thin film) νmax 2961, 2927, 2851, 1080, 764, 658 cm-1; 1H and 13C NMR data, see Tables 1 and 2; HR-APCIMS m/z 390.9890, 392.9868, 394.9847 [M+H]+ (53:100:50) (calcd. for C15H2179Br2O2, 390.9903, C15H2179Br81BrO2, 392.9882, C15H2181Br2O2, 394.9862).

Thuwalenyne A (a4): Colorless oil; [α]20D+9 (c 1.16, CHCl₃); UV (CHCl₃) λmax (log ε) 242 (3.14); IR (thin film) νmax 3292, 2959, 2925, 2855, 1112, 1093, 1059, 998 cm-1; 1H and 13C NMR data, see Tables 1 and 2; HR-APCIMS m/z 390.9889, 392.9865, 394.9843 [M+H]+ (50:100:50) (calcd. for C15H2179Br2O2, 390.9903, C15H2179Br81BrO2, 392.9882, C15H2181Br2O2, 394.9862).

Thuwalallene D (a5): Colorless oil; [α]20D −62 (c 0.38, CHCl₃); UV (CHCl₃) λmax (log ε) 241 (3.08); IR (thin film) νmax 3434, 2925, 2857, 1730, 1108, 1059, 660 cm-1; 1H and 13C NMR data, see Tables 1 and 2; HR-APCIMS m/z 442.9602, 444.9579, 446.9557 and 448.9528 [M+H]+ (51:100:49:15) (calcd. for C15H2279Br235ClO3, 442.9619, C15H2279Br81Br35ClO3, C15H2279Br237ClO3, 444.9518, C15H2281Br235ClO3, C15H2279Br81Br37ClO3, 446.9578, C15H2281Br237ClO3, 448.9548).

Thuwalenyne B (a6): Colorless oil; [α]20D −7 (c 0.59, CHCl₃); UV (CHCl₃) λmax (log ε) 242 (3.08); IR (thin film) νmax 3300, 2959, 2925, 2882, 2857, 1437, 1108, 1100, 1057, 616 cm-1; 1H and 13C NMR data, see Tables 1 and 2; HR-APCIMS m/z 390.9891, 392.9868, 394.9846 [M+H]+ (52:100:48) (calcd. for C15H2179Br2O2, 390.9903, C15H2179Br81BrO2, 392.9882, C15H2181Br2O2, 394.9862).

Thuwalenyne C (a7): Colorless oil; [α]20D −15 (c 0.08, CHCl₃); UV (CHCl₃) λmax (log ε) 240 (3.61); IR (thin film) νmax 3287, 2926, 1717, 1076; 1H and 13C NMR data, see Tables 1 and 2; HR-APCIMS m/z 313.0797, 315.0779 [M+H]+ (100:98) (calcd. for C15H2279BrO2, 313.0803, C15H2281BrO2, 315.0783).

Thuwalallene E (a8): Colorless oil; [α]20D −150 (c 0.45, CHCl₃); UV (CHCl₃) λmax (log ε) 243 (2.84); IR (thin film) νmax 2967, 2933, 2880, 2853, 1711, 1063, 800, 660 cm-1; 1H and 13C NMR data, see Tables 1 and 2; HR-APCIMS m/z 390.9896, 392.9877, 394.9857 [M+H]+ (53:100:47) (calcd. for C15H2179Br2O2, 390.9903, C15H2179Br81BrO2, 392.9882, C15H2181Br2O2, 394.9862).

### 1.4. Cell Culture

Mouse macrophage cell line RAW 264.7 was cultured in DMEM medium (cat. #21885-025, Gibco) supplemented with 10% heat inactivated fetal bovine serum (cat. #10270-106, Gibco) and 1% penicillin-streptomycin (cat. #15070-063, Gibco). Cells were cultured in 37° C. and 5% CO2. Each compound was diluted in Carbowax™ 400+10% ethanol (cat. #1.00983, Sigma), used also as control solvent. Final concentration in culture was 0.1% v/v carbowax and 0.01% v/v ethanol. RAW 264.7 macrophages were activated using 100 ng/mL lipopolysaccharide (LPS) (L2630, Sigma). In IC₅₀ determination experiments, macrophages were pre-treated for 1 h with the respective compound prior to LPS stimulation.

### 1.5. Nitric Oxide Measurement

30×104 RAW 264.7 mouse macrophages were plated in 24-well plates over-night with 0.5 mL complete medium. Cells were pretreated for 1 h with the respected compound concentration and then stimulated with 100 ng/mL LPS (L2630, Sigma) for 48 h. The amount of nitrite, an oxidative product of NO, was measured in culture supernatant of each sample using the Griess reaction. 100 μL of supernatant was mixed with 100 μl of sulfanilamide solution (1% sulfanilamide in 5% H₃PO₄) and incubated for 5 min at room temperature. Then, 100 μL of NED solution (0.1% N-1-naphtylethylenediamine dihydrochlorite in H₂O) was added and the absorbance was measured in an automated microplate reader (Infinate 200 PRO, Tecan) at 540 nm. Nitrite concentration was calculated using a sodium nitrite standard curve. All incubations were performed in the dark.

### 1.6. MTT Measurement

3.5×103 RAW 264.7 mouse macrophages were seeded in 96-well plate and cultured overnight. Cells were subsequently treated with the respective compound concentration and incubated for 24, 48 and 72 h. Number of cells was measured prior to treatment and used as normalisation control. Thiazolyl Blue Tetrazolium Bromide (MTT) (A2231.001, Applichem) was added to the cells in a final concentration of 0.5 mg/mL and then cells were incubated at 37° C. and 5% CO₂ for 4 h. The supernatant was discarded and cells were lysed with 2-propanol (33539, Honeywell) with 0.4% HCl (30721, Sigma). The absorbance of each sample was measured in an automated microplate reader (Infinite 200 PRO, Tecan) at 600 nm. The average OD of each treated sample was normalized to the OD of the control sample and statistical analysis was performed using Graphpad Prism 7.0.

### 1.7. Statistical Analysis

All data are presented as mean±SEM and as percentage in case of IC₅₀ evaluation. Statistical analysis was performed using Graphpad Prism 7.0. D'Agostino & Pearson, Shapiro Wilk and KS tests were used to evaluate normality. In case of normality, one-way ANOVA was performed, whereas in all other cases the non-parametric Kruskal-Wallis test was used. Differences with a P value <0.05 are considered significant (*indicates P<0.05, **indicates P<0.01, ***indicates P<0.001).

2. Results and Discussion

### 2.1. Structure Elucidation of the Isolated Metabolites

The organic extract of specimens of a Saudi Arabian population of the red alga Laurencia was subjected to a series of chromatographic separations to yield 11 compounds (a1-a11), including eight new C15 acetogenins (a1-a8) and three previously reported metabolites, which were identified as cis-maneonene D (a9) [11], thyrsiferol (a10) [12] and 23-acetyl-thyrsiferol (a11) [13] by comparison of their spectroscopic and physical characteristics with those reported in the literature.

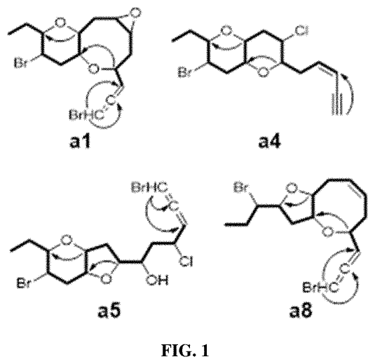

Thuwalallene A (a1) was isolated as colorless oil with the molecular formula C₁₅H₂₀O₃Br₂, as indicated by its HR-APCIMS and NMR data. The HSQC and HMBC spectra confirmed the presence of fifteen carbon atoms, corresponding to one non-protonated carbon, nine methines, four methylenes and one methyl (Table 1). A bromoallene moiety was evident from the chemical shifts of the allenic carbons at δC 201.5, 102.7 and 75.7, while the presence of seven deshielded methines bearing halogen or oxygen atoms at δC 83.7, 78.6, 76.3, 73.6, 52.7, 52.3 and 48.6 was observed. Additionally, in the 1H NMR spectrum (Table 2) presented signals for a methyl on a secondary carbon (δH 0.95) and seven methines resonating at δH 4.36, 4.11, 3.58, 3.36, 3.32, 3.19, and 3.09 attributed to protons of oxygenated or halogenated carbons. Since the allene moiety accounted for two of the five degrees of unsaturation, the molecular structure of 1 was determined as tricyclic. The cross-peaks observed in the COSY spectrum revealed a sole spin system extending from C-3 to C-15, placing the heteroatoms at C-4, C-6, C-7, C-9, C-10, C-12 and C-13. The HMBC correlations of H-4 to C-10 and H-7 to C-13 determined the presence of a tetrahydropyran and an oxocane ring, thus establishing the rare 4,10:9,13-bisepoxy core in the molecule (a1-a11). The third oxygen atom, in conjunction with the chemical shifts of C-6 and C-7 mandated the presence of an epoxy ring, thus completing the planar structure of metabolite a1. The relative configuration of the stereogenic centers of a1 was proposed on the basis of the key correlations displayed in the NOESY spectrum (FIG. 1) and the measured coupling constants. In particular, the coupling constants of H-12 (J=12.5, 10.3, 4.0 Hz) established its axial orientation. The NOE cross-peaks of H-12 with H-11α (δH 2.45), of H-13 with H-9 and of the latter with H-10, as well as of H-11β (δH 2.04) with H-9, H-10 and H-13 determined the cis fusion of the tetrahydropyran and oxocane rings and established the relative configuration at C-9, C-10, C-12 and C-13. The NOE enhancement of H-4 with H-10 determined the cis orientation of H-4 and H-10. Additionally, the NOE correlations of H-9 with H-8α (δH 2.59) and H-8β (δH 1.33), of H-8α with H-7, of H-5β (δH 1.65) with H-4 and H-8β and of H-5α (δH 2.25) with H-6 established the relative configuration at C-4, C-6, and C-7. According to the empirical rule proposed by Lowe about the absolute configuration of chiral allenes [14,15], the negative sign of the optical rotation measured for compound 1 was indicative of the 2R configuration of the bromoallene moiety. Thus, the configuration of metabolite 1 was established as 2R,4R*,6S*,7R*,9R*,10R*,12R*,13S*.

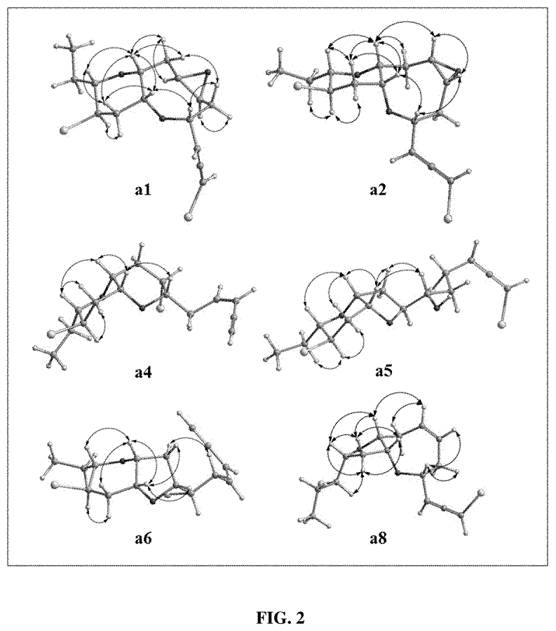

Thuwalallene B (a2), obtained as colorless oil, exhibited the same molecular formula as 1 according to its HR-APCIMS and NMR data. Compound a2 exhibited rather similar spectroscopic data to those of 1 (Table 1 and Table 2), showing that compounds a1 and a2 were stereoisomers. Indeed, characteristic correlations of a bromoallene moiety (δC201.9, 102.0 and 73.7), along with signals of seven heteroatom-bearing methines (δC82.7, 79.5, 76.2, 75.6, 52.4, 51.8 and 48.6), were observed for 2 in its HSQC and HMBC spectra. After thorough analysis of the homonuclear correlations observed in the COSY spectrum of a2, the same spin system extending from C-3 to C-15 was identified, while on the basis of the heteronuclear correlations displayed in the HMBC spectrum, ether linkages between C-4 and C-10, C-9 and C-13 and C-6 and C-7 were observed, as in the case of 1, confirming the same gross structure. The relative configuration of compound a2 was elucidated after detailed analysis of the NOE enhancements observed and the measured coupling constants (FIG. 2). As in the case of a1, the relative configurations at C-4, C-9, C-10, C-12 and C-13 were determined as 4R*,9R*,10R*,12R*,13S* on the basis of the NOE interactions of H-4 and H-10, of H-12 and both H-11α (δH 2.57) and H-14b (δH 1.49), of H-9 and both H-10 and H-13, and of H-11β (δH 2.08) with H-9, H-10 and H-13. In contrast, the NOE correlations of H-9 with H-8β (δH 2.39) and H-7 and of H-6 with both H-4 and H-7 established the relative configuration at C-6 and C-7 as 6R*,7S*. The negative sign of the optical rotation measured for compound 2, which was determined as the 6,7-stereoisomer of 1, was again indicative of the 2R configuration of the bromoallene moiety.

Thuwalallene C (a3) was obtained as colorless oil. Its molecular formula was deduced as C₁₅H₂₀O₃Br₂ on the basis of its HR-APCIMS and NMR data. The NMR spectroscopic data of a3 (Table 1 and Table 2) showed a close resemblance to those of a1 and a2. The main difference was the absence of the two oxygenated methines attributed to H-6 and H-7, while it was clear the replacement by two olefinic methines resonating at δH 5.71 and 5.82, which were assigned on the basis of the COSY cross-peaks observed. The relative configuration of a3, designated as thuwalallene C, was determined mainly on the basis of the enhancements observed in its NOESY spectrum, in close resemblance to those of metabolite a2, as 4R*,9R*,10R*,12R*,13S*. The absolute configuration of the bromoallene functionality was established as 2R on the basis of the negative sign of the measured optical rotation.

Thuwalenyne A (a4) was isolated as colorless oil and exhibited the molecular formula C₁₅H₂₀O₂Br₂, as determined on the basis of its HR-APCIMS and NMR data (Table 1 and Table 2). The HSQC and HMBC spectra of a4 displayed correlations indicative of 15 carbons corresponding to one non-protonated carbon, nine methines, four methylenes, and one methyl. Among them, four carbons were bonded to an oxygen atom (δC70.7, 75.6, 78.3 and 82.1) and two were halogenated (δC46.4 and 47.1). In addition, the chemical shift of the quaternary carbon at δC79.9, along with the resonances of three tertiary carbons at δC82.1, 111.1 and 140.2 were indicative of a terminal-enyne moiety. The geometry of the double bond was determined as Z due to the coupling constant value (J=10.7 Hz) between the olefinic methines H-3 and H-4, as also suggested by the chemical shift of the acetylenic proton H-1 (δ3.11). The COSY cross-peaks readily identified the extended spin system spanning from C-3 to C-15, while the HMBC correlations of H-6 to C-10 and of H-9 to C-13 established a 2,7-dioxabicyclo[4.4.0]decane ring system (FIG. 1). The relative configuration of the asymmetric centers of 4 was determined on the basis of the observed NOE enhancements and measured coupling constants (FIG. 2). The strong NOE interactions of H-6/H-10, H-10/H-9, and H-9/H-13 revealed the cis fusion of the two pyran rings and the coplanar orientation of H-6, H-9, H-10 and H-13. Furthermore, the NOE enhancements of H-9/H-11β and of H-11α/H-12 determined the relative configuration at C-12, whereas the coupling constants measured for H-12 (J=12.1, 10.0, 4.4 Hz) established its axial orientation. The fact that H-7 appeared as a broad singlet demonstrates its equatorial orientation, which in conjunction with its NOE interaction with H-6 established its relative configuration. Thus, the relative configuration of metabolite a4 was determined as 6S*,7S*,9R*,10R*,12R*,13S*.

Thuwalallene D (a5) was obtained as colorless oil. The HR-APCIMS spectrum exhibited isotopic pseudomolecular ion peaks [M+H]+ at m/z 442.9602, 444.9579, 446.9557 and 448.9528 with a ratio of 51:100:49:15, characteristic for the presence of one chlorine and two bromine atoms in the molecule. Based on the HR-APCIMS and NMR data, the molecular formula of a5 was deduced as C₁₅H₂₁Br₂ClO₃. The structural elements of a5 included a bromoallene functionality (δC200.5, 102.1, 75.1), seven halogenated or oxygenated methines (δC81.5, 80.2, 78.3, 77.7, 70.6, 54.5, 47.4), four methylenes (δC40.9, 38.4, 35.8, 26.1) and a methyl (δC8.8). The cross-peaks observed in the COSY spectrum, in combination with the HMBC correlation of H-7 with C-10 and H-9 with C-13 established a 7,10:9,13-bisepoxy core and placed the chlorine atom at C-4, a hydroxy group at C-6 and the second bromine atom at C-12 (FIG. 1). The strong NOE enhancement between H-9 and H-10 suggested the cis fusion of the two rings. Moreover, the coupling constants of H-12 (J=11.8, 10.2, 4.3 Hz) established its axial orientation, while the NOE interactions of H-9/H-13, H-11α/H-12, H-11β/H-9 and H-12/H-14b (δH 1.52) defined the relative configurations of the chiral centers C-9, C-10, C-12 and C-13 of the pyran ring. The NOE cross-peaks of H-8β with both H-6 and H-10, as well as the weak, albeit observable, correlation of H-6 with H-10 suggested the relative configuration at C-7. The relative configuration at C-4 and C-6 could not be safely assigned based solely on NOE data. On this basis, the relative configuration of the chiral centers of a5 was determined as 7S*,9R*,10R*,12R*,13S* (FIG. 2), while the negative sign of the optical rotation measured indicated the 2R configuration of the bromoallene.

Thuwalenyne B (a6) was obtained as colorless oil. Its molecular formula was deduced as C₁₅H₂₀O₂Br₂ on the basis of HR-APCIMS and NMR data (Table 1 and Table 2). As in the case of a5, analysis of the correlations displayed in COSY, HSQC and HMBC spectra of a6 established the same 7,10:9,13-bisepoxy core, with the main difference being the presence of an -enyne terminus, evident from the chemical shift of the quaternary carbon at δC81.8, along with the resonances of three tertiary carbons at δC141.6, 110.5 and 80.0, instead of a bromoallene moiety. The geometry of the 1,2-disubstituted double bond was defined as Z due to the coupling constant (J=10.7 Hz) measured between the olefinic H-3 and H-4, corroborated by the chemical shift of the acetylenic proton H-1 (δ3.11). The relative configuration of compound 6 was elucidated after thorough analysis of the NOE enhancements and the observed coupling constants (FIG. 2). The NOE interaction between H-9 and H-10 determined the cis fusion of the tetrahydropyran and tetrahydrofuran rings. The axial orientation of H-12 was determined on the basis the observed coupling constants (J=11.8, 10.2, 4.5 Hz), while the NOE interactions of H-9/H-13, H-9/H-11β and H-11α/H-12 established the coplanar orientation of H-9 and H-13. The NOE enhancements of H-10 with both H-7 and H-8β, as well as of H-5a (δH 3.02) with H-8a determined the relative configuration at C-7. Thus, the configuration of compound a6, designated as thuwalenyne B, was assigned as 7R*,9R*,10R*,12R*,13S*.

Thuwalenyne C (a7), obtained as colorless oil, possessed the molecular formula C₁₅H₂₁BrO₂, as determined by the HR-APCIMS and NMR data (Table 1 and Table 2). The presence of -enyne functionality, as observed from the 1H and 13 C chemical shifts, along with the isolated double bond accounted for four of the five degrees of unsaturation, thus indicating that a7 was a monocyclic C15 acetogenin. The HMBC correlation of H-9 with C-13 led to the identification of one tetrahydropyran ring in the structure of a7, while the COSY cross-peaks placed the isolated double bond between C-6 and C-7, as well as a hydroxy group at C-10 and the bromine atom at C-12. Thorough analysis of the NOE enhancements in combination with the observed coupling constants led to the identification of the relative configuration of a7. The coupling constant of H-12/H-13 (J=9.9 Hz) indicated their diaxial orientation, whereas the NOE interactions of H-9/H-13, H-11α/H-12 and H-9/H-11β established the relative configuration at C-9 and C-13. The equatorial orientation of H-10, and thus the relative configuration at C-10, was established based on the small coupling constants of H-10 (brs). The geometry of the 1,2-disubstituted double bond of the—enyne moiety was determined as Z according to the coupling constant (J=10.9 Hz) between H-3 and H-4 and the chemical shift of the acetylenic proton resonating at δ3.10. Furthermore, the geometry of the Δ6 double bond was identified as Z due to the resonance of the doubly allylic methylene carbon C-5 at δc 28.0 [16], as well as the NOE cross-peak of H2-5/H2-8. Thus, the relative configuration of a7 was established as 9R*,10R*,12R*,13S*.

Thuwalallene E (a8), isolated as colorless oil, displayed the molecular formula C15H₂₀O₂Br₂, as derived from the HR-APCIMS and NMR data. The NMR spectroscopic features of a8 were rather similar to those of a3 (Table 1 and Table 2). A single spin system was identified based on the COSY correlations, spanning from C-3 to C-15. As in the case of a3, the HMBC correlation of H-4 to C-10 identified an oxocane ring. However, the HMBC correlation of H-9 with C-12 instead of C-13 established a tetrahydrofuran instead of a tetrahydropyran as the second ring of the bicyclic system of a8 (FIG. 1). The NOE enhancements of H-4/H-5β, H-4/H-6, H-7/H-8β, H-7/H-9, H-9/H-11β, H-9/H-12, H-10/H-11β and H-11β/H-12, as well as of H-10/H-11α, H-11α/H-12 and H-11α/H-13 established the cis fusion of the 4,10:9,12-bisepoxy ring system and the coplanar orientation of H-4, H-9, H-10 and H-12, thus determining the relative configuration at C-4, C-9, C-10, C-12 as 4R*,9R*,10R*,12R* (FIG. 1). Furthermore, the 2R configuration of the bromoallene was assigned on the basis of the negative sign of its measured optical rotation.

TABLE 1
¹³C NMR data (δ in ppm) in CDCl₃ of compounds a1-a8.

| Position | a1 ^{1,2} | a2 ^{1,3} | a3 ⁴ | a4 ^{1,3} | a5 ^{1,3} | a6 ^{1,5} | a7 ^{1,5} | a8 ⁴ |
|---|---|---|---|---|---|---|---|---|
| 1 | 75.7 | 73.7 | 73.8 | 82.1 | 75.1 | 80.0 | 80.7 | 74.2 |
| 2 | 201.5 | 201.9 | 201.0 | 79.9 | 200.5 | 81.8 | 78.6 | 200.9 |
| 3 | 102.7 | 102.0 | 103.2 | 111.1 | 102.1 | 110.5 | 108.0 | 103.8 |
| 4 | 73.6 | 75.6 | 78.0 | 140.2 | 54.5 | 141.6 | 142.9 | 79.9 |
| 5 | 31.2 | 36.3 | 34.2 | 35.6 | 40.9 | 34.2 | 28.0 | 34.5 |
| 6 | 52.3 | 52.4 | 129.6 | 78.3 | 70.6 | 55.6 | 127.9 | 128.9 |
| 7 | 52.7 | 51.8 | 129.4 | 46.4 | 81.5 | 80.9 | 126.0 | 130.1 |
| 8 | 32.6 | 29.9 | 30.1 | 36.5 | 35.8 | 36.1 | 29.1 | 28.8 |
| 9 | 76.3 | 76.2 | 79.7 | 70.7 | 77.7 | 76.1 | 79.7 | 85.9 |
| 10 | 78.6 | 79.5 | 77.7 | 75.6 | 78.3 | 79.4 | 69.1 | 83.1 |
| 11 | 43.5 | 42.1 | 42.7 | 40.5 | 38.4 | 37.7 | 43.0 | 39.7 |
| 12 | 48.6 | 48.6 | 49.3 | 47.1 | 47.4 | 47.4 | 47.6 | 81.7 |
| 13 | 83.7 | 82.7 | 83.2 | 82.1 | 80.2 | 80.6 | 83.5 | 61.9 |
| 14 | 26.0 | 26.0 | 26.4 | 25.7 | 26.1 | 26.0 | 25.5 | 28.1 |
| 15 | 9.2 | 9.3 | 9.6 | 7.9 | 8.8 | 8.8 | 9.1 | 11.6 |

¹ Chemical shifts were determined through HMBC correlations.
² Recorded at 100 MHz.
³ Recorded at 150 MHz.
⁴ Recorded at 175 MHz.
⁵ Recorded at 237.5 MHz.

TABLE 2
¹H NMR data(δ in ppm, J in Hz) in CDCl₃ of compounds a1-a8.

| Position | a1 ¹ | a2 ² | a3 ³ | a4 ² | a5 ² | a6 ⁴ | a7 ⁴ | a8 ³ |
|---|---|---|---|---|---|---|---|---|
| 1 | 6.11 | 6.06 | 6.02 | 3.11 | 6.13 | 3.11 d | 3.10 m | 6.06 |
| | dd | dd | dd | brs | dd | (1.6) | | dd |
| | (5.6, 2.6) | (5.5, 2.1) | (5.6, 1.8) | | (5.7, 1.7) | | | (5.7, 2.5) |
| 3 | 5.56 | 5.52 | 5.52 | 5.55 br | 5.58 | 5.60 br dd | 5.46 m | 5.45 |
| | dd | dd | dd | d | dd | (10.7, 1.6) | | dd |
| | (5.6, 4.5) | (5.5, 5.5) | (5.6, 5.6) | (10.7) | (6.9, 5.7) | | | (5.7, 4.5) |
| 4 | 4.36 m | 4.18 | 4.01 m | 6.07 | 4.82 m | 6.15 ddd | 5.93 | 3.90 m |
| | | ddd | | ddd | | (10.7, 7.0, | ddd | |
| | | (11.0, 5.5, | | (10.7, 7.3, | | 7.0) | (10.9, 7.4, | |
| | | 2.1) | | 7.3) | | | 7.4) | |
| 5 | α 2.25 | α 1.67 | α 2.56 | a 2.61 | a 1.99 | a 3.02 | 3.10 m | α 2.52 |
| | dd | ddd | m | m | m | dddd | | ddd |
| | (14.3, 4.5) | (14.6, | β 2.16 | b 2.73 | b 1.74 | (16.0, 7.0, | | (15.1, |
| | | 11.0, | brdd | ddd | ddd | 2.9, 1.6) | | 10.4, |
| | β 1.65 | 9.5) | (14.2, | (14.4, | (13.4, | b 2.80 ddd | | 6.0) |
| | m | β 2.36 | 8.1) | 7.3, | 11.0, | (16.0, 7.0, | | β 2.16 |
| | | m | | 7.3) | 1.7) | 7.0) | | m |
| 6 | 3.09 | 3.00 m | 5.82 m | 3.30 m | 3.73 | 4.13 m | 5.48 m | 5.76 m |
| | ddd | | | | ddd | | | |
| | (10.8, | | | | (11.0, | | | |
| | 4.5, | | | | 6.1, | | | |
| | 4.5) | | | | 2.1) | | | |
| 7 | 3.19 | 2.87 | 5.71 m | 4.02 | 4.11 m | 4.16 m | 5.47 m | 5.79 m |
| | ddd | ddd | | brs | | | | |
| | (9.3, | (11.4, | | | | | | |
| | 4.5, | 3.7, | | | | | | |
| | 4.5) | 3.7) | | | | | | |
| 8 | α 2.59 | α 1.55 | α 2.59 | α 2.53 | α 2.05 | α 1.99 dd | 2.35 m | α 2.63 |
| | ddd | m | m | brd | brdd | (14.4, 4.3) | | m |
| | (14.6, | β 2.39 | β 2.23 | (15.7) | (13.3, | β 2.23 ddd | | β 2.24 |
| | 5.2, | m | m | β 2.31 | 6.2) | (14.4, 9.1, | | m |
| | 4.5) | | | ddd | β 1.82 | 5.2) | | |
| | β 1.33 | | | (15.7, | ddd | | | |
| | ddd | | | 4.3, | (13.3, | | | |
| | (14.6, | | | 4.3) | 9.6, | | | |
| | 9.3, | | | | 3.9) | | | |
| | 1.6) | | | | | | | |
| 9 | 3.58 | 3.70 | 3.53 | 3.59 | 4.12 m | 4.06 dd | 3.45 | 3.84 |
| | brd | ddd | brdd | brs | | (5.2, 1.7) | ddd | ddd |
| | (5.2) | (11.0, | (11.0, | | | | (7.3, | (11.3, |
| | | 5.3, | 5.3) | | | | 7.3, | 7.7, |
| | | 1.1) | | | | | 0.6) | 3.8) |
| 10 | 3.36 m | 3.55 | 3.63 | 3.48 | 3.91 | 3.77 m | 3.68 | 3.89 m |
| | | brs | brs | brs | brs | | brs | |
| 11 | α 2.47 | α 2.57 | α 2.52 | α 2.62 | α 2.70 | α 2.75 | α 2.57 | α 2.10 |
| | ddd | ddd | ddd | m | ddd | ddd (14.6, | ddd | m |
| | (12.7, | (12.9, | (13.3, | β 2.08 | (14.5, | 4.5, 2.3) | (13.7, | β 2.35 |
| | 4.2, | 3.6, | 3.5, | m | 4.3, | β 2.16 ddd | 4.5, | ddd |
| | 4.2) | 3.6) | 3.5) | | 2.4) | (14.6,11.8, | 3.6) | (14.8, |
| | β 2.04 | β 2.08 | β 1.99 | | β 2.13 | 3.7) | β 2.06 | 8.9, |
| | ddd | ddd | ddd | | ddd | | m | 6.4) |
| | (12.7, | (12.9, | (13.3, | | (14.5, | | | |
| | 12.7, | 12.9, | 13.3, | | 11.8, | | | |
| | 2.8) | 3.6) | 3.2) | | 3.5) | | | |
| 12 | 4.11 | 4.01 | 4.02 m | 4.16 | 3.96 | 4.04 ddd | 3.98 | 4.00 |
| | ddd | ddd | | ddd | ddd | (11.8, 10.2, | ddd | ddd |
| | (12.7, | (12.9, | | (12.1, | (11.8, | 4.5) | (12.3, | (8.9, |
| | 10.3, | 10.0, | | 10.0, | 10.2, | | 9.9, | 8.9, |
| | 4.2) | 3.6) | | 4.4) | 4.3) | | 4.5) | 4.5) |
| 13 | 3.32 | 3.28 | 3.25 | 3.33 | 3.28 | 3.29 ddd | 3.33 | 4.05 |
| | ddd | ddd | ddd | ddd | ddd | (10.2, 7.4, | ddd | ddd |
| | (10.3, | (10.0, | (9.3, | (10.0, | (10.2, | 2.6) | (9.9, | (8.9, |
| | 7.9, | 10.0, | 9.3, | 6.0, | 7.8, | | 9.9, | 8.9, |
| | 2.5) | 2.3) | 2.0) | 2.8) | 2.4) | | 2.1) | 2.6) |
| 14 | a 1.98 | a 2.01 | a 2.01 | a 1.87 | a 1.97 | a 1.94 dqd | a 2.04 | a 2.16 |
| | dqd | dqd | dqd | dqd | m | (14.7, 7.4, | m | m |
| | (14.8, | (15.2, | (14.9, | (14.3, | b 1.52 | 2.6) | b 1.49 | b 1.73 |
| | 7.3, | 7.4, | 7.3, | 7.3, | m | b 1.57 dqd | m | m |
| | 2.5) | 2.3) | 2.0) | 2.8) | | (14.7, 7.4, | | |
| | b 1.58 | b 1.49 | b 1.50 | b 1.76 | | 7.4) | | |
| | m | m | m | m | | | | |
| 15 | 0.95 t | 0.93 t | 0.96 t | 0.98 t | 0.93 t | 0.92 t (7.4) | 0.96 t | 1.06 t |
| | (7.3) | (7.4) | (7.3) | (7.3) | (7.4) | | (7.4) | (7.3) |

¹ Recorded at 400 MHz.
² Recorded at 600 MHz.
³ Recorded at 700 MHz.
⁴ Recorded at 950 MHz.

### 2.2. Evaluation of the Anti-inflammatory Activity of the Isolated Metabolites

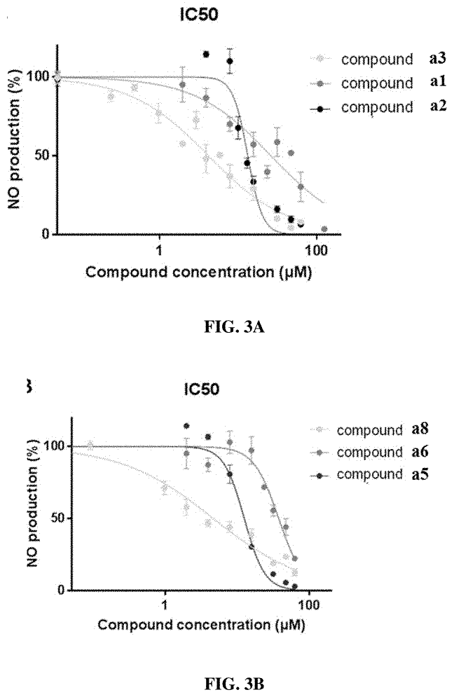

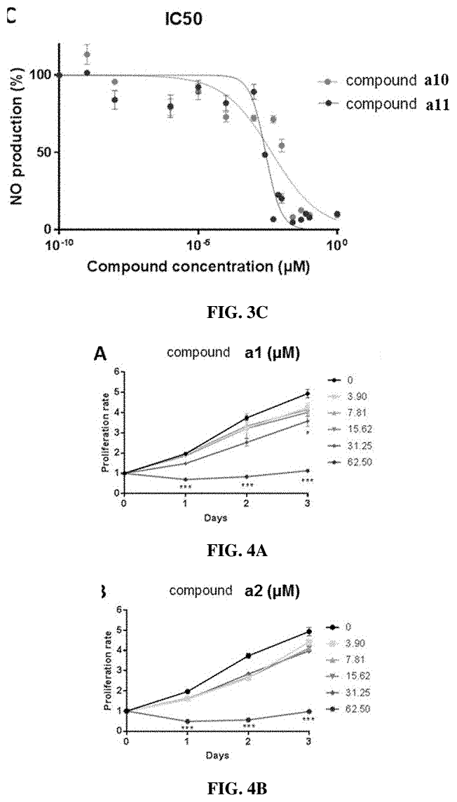

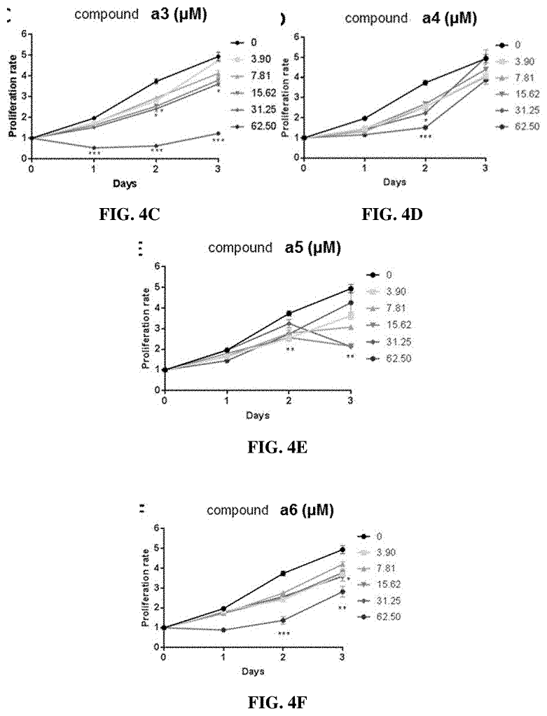

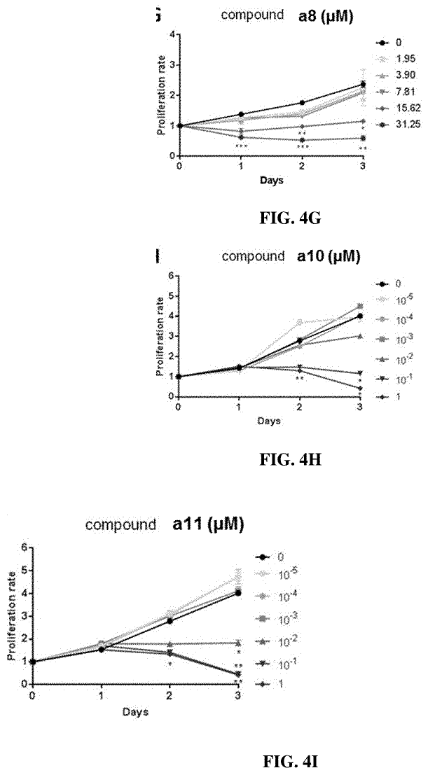

Compounds a1-a6, a8, a10 and a11 were evaluated for their anti-inflammatory activity using Griess reaction to quantify nitric oxide release in response to TLR4 stimulation in macrophages, being the main pro-inflammatory factor. The IC₅₀ values for the inhibition of NO production were determined in LPS-treated RAW 264.7 cells in comparison to Carbowax 400 (Table 3 and FIG. 3). To verify that the anti-inflammatory activity observed was not due to cytotoxic events, the potential cytotoxicity of compounds a1-a6, a8, a10 and a11 was evaluated using the MTT assay following 24, 48 and 72 h of treatment in RAW 264.7 macrophages (Table 3 and FIG. 4).

Compounds a1 and a2 exhibited IC₅₀ values of 26.03 μM and 13.23 μM, with cytotoxicity higher than 15.62 μM and 31.25 μM, respectively, at 72 h following treatment. Compound a3, although it displayed structural similarities to a1 and a2, showed a lower IC₅₀ value of 4.181 μM and cytotoxicity higher than 15.62 μM at 72 h of treatment. Compound a4 did not exhibit anti-inflammatory activity or cytotoxicity at all concentrations tested. Compound a6 exhibited weak anti-inflammatory activity with an IC₅₀ value of 37.39 μM that was primarily attributed to its cytotoxic activity since it exhibited toxicity to RAW 264.7 macrophages at concentrations higher than 15.62 μM. On the contrary, compound a5 displayed anti-inflammatory activity with an IC₅₀ value of 12.41 μM but significant cytotoxicity only at concentrations above 15.62 μM 72 h following treatment. Compound a8 exhibited potent anti-inflammatory activity with an IC₅₀ value of 3.98 μM, whereas it was not cytotoxic in concentrations below 7.81 μM 72 h following treatment. The most potent anti-inflammatory activity was observed for compounds a10 and a11, which were active with IC₅₀ values of 4.387 nM and 2.633 nM, respectively. Compound a10 exhibited significant cytotoxicity at concentrations above 100 nM, whereas compound a11 at concentrations above 10 nM, indicating that the potent anti-inflammatory activity was not due to their cytotoxicity. This is the first report on the anti-inflammatory activity of C₁₅ acetogenins.

TABLE 3
IC₅₀ values (in μM) for inhibition of NO production
and cytotoxicity for compounds a1-a6, a8, a10 and a11.

| | | Cytotoxicity |
|---|---|---|
| Compound | Inhibition of NO production | (at 72 h) |
| a1 | 26.03 ± 3.731 | >15.62 |
| a2 | 13.23 ± 0.5655 | >31.25 |
| a3 | 4.181 ± 0.481 | >15.62 |
| a4 | >62.5 | >62.5 |
| a5 | 12.41 ± 1.037 | >15.62 |
| a6 | 37.39 ± 2.514 | >15.62 |
| a8 | 3.98 ± 0.6016 | >7.81 |
| a10 | 4.387 × 10⁻³ ± 0.8505 × 10⁻³ | >0.1 |
| a11 | 2.633 × 10⁻³ ± 0.2378 × 10⁻³ | >0.01 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### REFERENCES

1. AlgaeBase. Available online: http://www.algaebase.org/(accessed on May 6, 2019).
2. Harizani, et al., J., Eds.; Springer International Publishing: Switzerland, 2016; Volume 102, pp. 91-252.
3. Paul, et al. Nat. Prod. Rep. 2011, 28, 345-387.
4. MarinLit. A database of the marine natural products literature. Available online: http://pubs.rsc.org/marinlit/(accessed on May 6, 2019).
5. Blunt, et al. Nat. Prod. Rep. 2015, 32, 116-211.
6. Kamada, et al. Nat. Prod. Res. 2019, 33, 464-471.
7. Perdikaris, et al. Org. Lett. 2019, 21, 3183-3186.
8. Daskalaki, et al. Mar. Drugs 2019, 17, 97-111.
9. Kokkotou, et al. Phytochemistry 2014, 108, 208-219.
10. Lhullier, et al. J. Nat. Prod. 2010, 73, 27-32.
11. Ayyad et al. Chem. Pharm. Bull. 2011, 59, 1294-1298.
12. Blunt, et al. Tetrahedron Lett. 1978, 1, 69-72.
13. Suzuki, et al. Tetrahedron Lett. 1985, 26, 1329-1332.
14. Lowe, et al. J. Chem. Soc., Chem. Commun. 1965, 411-413.
15. Elsevier, et al. J. Org. Chem. 1985, 50, 364-367.
16. de Silva, et al. J. Org. Chem. 1983, 48, 395-396.

We claim:

1. A composition comprising a compound having a structure of Formula (I): 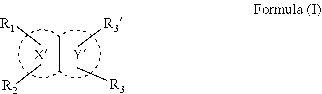
wherein X′ is a C₄ five-membered heterocyclic group or a C₅ six-membered heterocyclic group;
wherein Y′ is a C₄ five-membered heterocyclic group, a C₅ six-membered heterocyclic group, or a C₇ eight-membered heterocyclic group;
wherein R₁ and R₂ are independently absent, a halogen, or a substituted or unsubstituted alkyl group;
wherein R₃ and R₃′ are independently absent, a halogen, a hydroxyl group, a thiol group, an amino group, 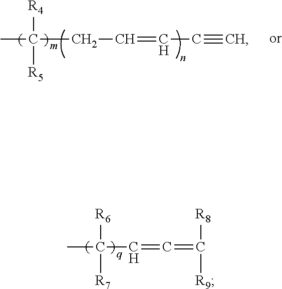
wherein R₄-R₉ are independently a hydrogen, a halogen, an hydroxyl group, a thiol group, or an amino group;
wherein each of m, n, and q is an integer from 0 to 3; and
wherein when R₁ and/or R₂ are independently a substituted alkyl group, the substituent is a halogen, an hydroxyl group, a thiol group, or an amino group, and optionally, an excipient.

2. The composition of claim 1 wherein the compound has a structure of Formula (II): 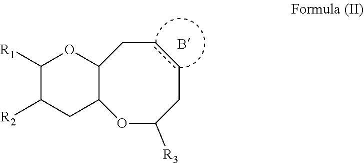
wherein B′ is absent or an epoxide group;
wherein R₁ and R₂ are independently a halogen or an unsubstituted alkyl group;
wherein R₃ is 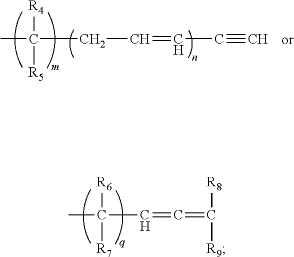
wherein R₄-R₉ are independently a hydrogen, a halogen, an hydroxyl group, a thiol group, or an amino group; and
wherein each of m, n, and q is an integer from 0 to 3.

3. The composition of claim 2,
wherein R₃ is 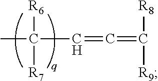
wherein R₆-R₉ are independently a hydrogen or a halogen; and
wherein q is an integer from 0 to 3.

4. The composition of claim 2 wherein the compound has a structure of Formula (II′): 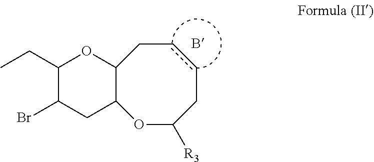
wherein B′ and R₃ are as defined in the base claim(s).

5. The composition of claim 1, wherein the composition has a structure of Formula (III): 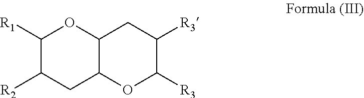
wherein R₁ and R₂ are independently a halogen or an unsubstituted alkyl group;
wherein R₃ and R₃′ are independently a halogen, 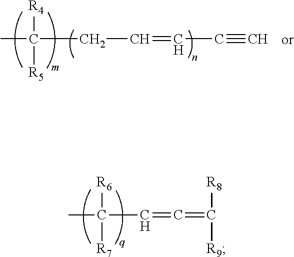
wherein R₄-R₉ are independently a hydrogen, a halogen, an hydroxyl group, a thiol group, or an amino group; and
wherein each of m, n, and q is an integer from 0 to 3.

6. The composition of claim 5,
wherein R₃′ is a halogen;
wherein R₃ is 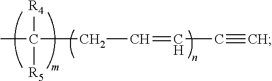
wherein R₄ and R₅ are independently a hydrogen or a halogen; and
wherein m and n are independently an integer from 0 to 3.

7. The composition of claim 5 wherein the compound has a structure of Formula (III′): 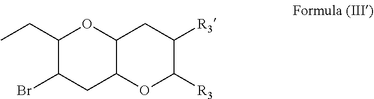
wherein R₃ and R₃′ are as defined in the base claim(s).

8. The composition of claim 1 wherein the compound has a structure of Formula (IV): 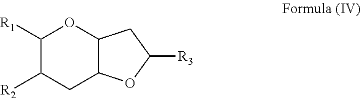
wherein R₁ and R₂ are independently a halogen or an unsubstituted alkyl group;
wherein R₃ is 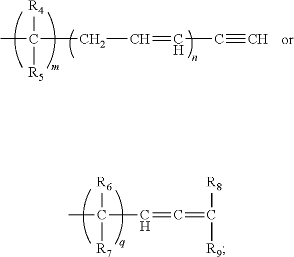
wherein R₄-R₉ are independently a hydrogen, a halogen, a hydroxyl group, a thiol group, or an amino group; and
wherein each of m, n, and q is an integer from 0 to 3.

9. The composition of claim 8,
wherein R₃ is 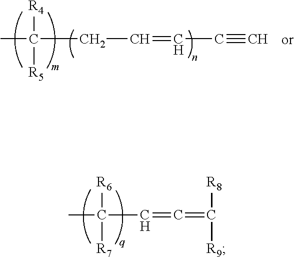
wherein R₄-R₉ are independently a hydrogen, a halogen, or a hydroxyl group; and
wherein each of m, n, and q is an integer from 0 to 3.

10. The composition of claim 8, wherein the compound has a structure of Formula (IV′): 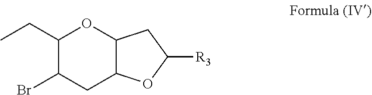
wherein R₃ is as defined in the base claim(s).

11. The composition of claim 1 wherein the compound has a structure of Formula (V): 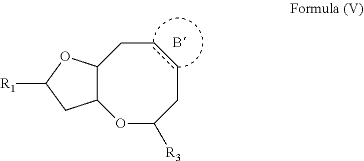
wherein B′ is absent or an epoxide group;
wherein R₁ is a halogen or a substituted alkyl group, wherein the substituent is a halogen, a hydroxyl group, a thiol group, or an amino group;
wherein R₃ is 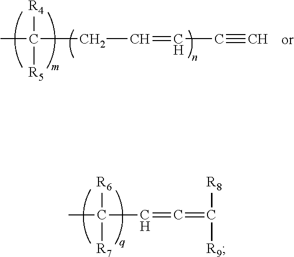
wherein R₄-R₉ are independently a hydrogen, a halogen, a hydroxyl group, a thiol group, or an amino group; and
wherein each of m, n, and q is an integer from 0 to 3.

12. The composition of claim 11,
wherein R₃ is 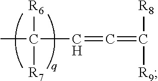
wherein R₆-R₉ are independently a hydrogen or a halogen; and
wherein q is an integer from 0 to 3.

13. The composition of claim 11, wherein the compound has a structure of Formula (V′): 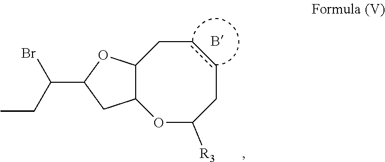
wherein B′ and R₃ are as defined for the base claim(s).

14. The composition of claim 1, wherein the compound has the structure of any one of a1-a8.

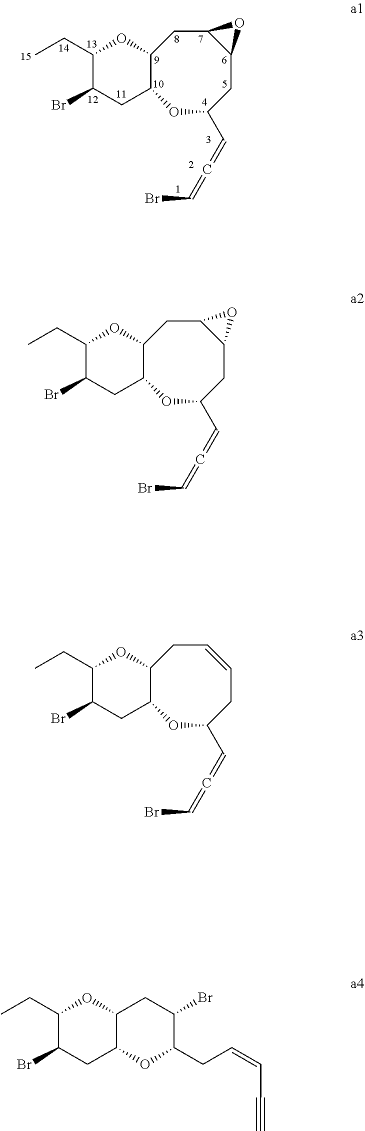

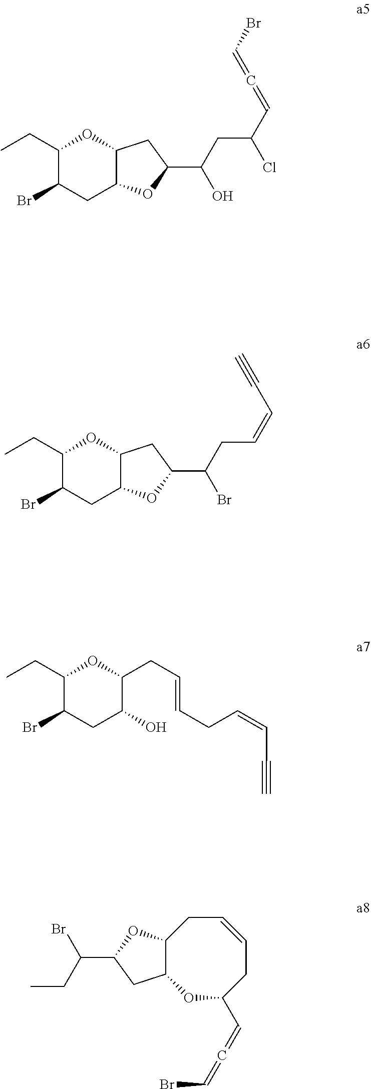

15. A method of making the compound of claim 1 comprising:
(i) extracting a fresh seaweed specimen with an extraction solvent to produce an organic extract;
(ii) subjecting the organic extract to liquid chromatography with a first mobile phase to yield a first panel of fractions, optionally (iii) subjecting one of the first panel of fractions to liquid chromatography with a second mobile phase to yield a second panel of fractions; and
(iv) purifying one of the first or the second panel of fractions using HPLC with a third mobile phase to yield a compound, optionally more than one compound.

16. The method of claim 15, wherein: (a) step (iii) is repeated for at least one time, at least two times, at least three times, at least five times, at least 10 times, or up to 20 times, wherein each repeat is performed prior to, simultaneously with, or subsequent to step (iv); and/or (b) each repeat of step (iii) is performed to separate the same fraction of the first panel of fractions or a different fraction of the first panel of fractions from the previous liquid chromatography separation; and each repeat of step (iii) is performed using the same mobile phase or a different mobile phase from the previous liquid chromatography separation.

17. The method of claim 16, wherein step (iv) is repeated at least one time, at least two times, at least three times, at least five times, at least 10 times, or up to 20 times, optionally wherein each repeat of step (iv) is performed to purify the same fraction of the first panel of fractions or a different fraction of the first panel of fractions from the previous purification, or
each repeat of step (iv) is performed to purify the same fraction of the second panel of fractions or a different fraction of the second panel of fractions from the previous purification; and wherein each repeat of step (iv) is performed using the same mobile phase or a different mobile phase from the previous purification.

18. A method for presenting, treating, or ameliorating one or more symptoms associated with an inflammation in a subject, comprising:
(i) administering to the subject an effective amount of the compound of any claim 14 to prevent, treat, or ameliorate one or more symptoms associated with inflammation in the subject.

19. The method of claim 18, wherein the compound is effective to inhibit nitric oxide (“NO”) production in the subject, and optionally wherein the compound has an IC₅₀ for NO production inhibition below about 40 μM, below about 35 μM, below about 30 μM, below about 28 μM, below about 25 μM, below about 20 μM, below about 15 μM, below about 10 μM, or below about 5 μM, against macrophage cells.

20. The method of claim 18, wherein the compound is administered to the subject by oral administration, parenteral administration, inhalation, mucosal, or topical administration, or a combination thereof, optionally, the method further comprising administering one or more additional active agents to the subject prior to, during, and/or subsequent to step (i).
